(19) United States
(12) Patent Application Publication
(10) Pub. No.: US 2023/0349899 A1
(43) Pub. Date: Nov. 2, 2023

(54) METHODS AND COMPOSITIONS RELATING TO ASSESSMENT FOR INFERTILITY

(71) Applicant: Microgenesis Corporation, San Francisco, CA (US)

(72) Inventors: Gabriela Lidia GUTIERREZ, Caba (AR); Maria Augustina AZPIROZ, Buenos Aires (AR)

(21) Appl. No.: 18/179,190

(22) Filed: Mar. 6, 2023

Related U.S. Application Data
(63) Continuation of application No. PCT/US2021/049604, filed on Sep. 9, 2021.
(60) Provisional application No. 63/076,690, filed on Sep. 10, 2020.

(51) Int. Cl. G01N 33/569; A61K 45/06; A61K 35/741; A61P 15/08

(52) U.S. Cl. CPC G01N 33/56911; A61K 45/06; A61K 35/741; A61P 15/08; G01N 2800/52

(57) ABSTRACT

Provided herein are methods and compositions for assessing infertility in an individual comprising detecting a bacteria, a miRNA, or both. Further provided herein are administering a therapy based on the assessment.

### CROSS-REFERENCE

This application is a continuation of International Patent Application No. PCT/US2021/049604, filed Sep. 9, 2021, which claims the benefit of U.S. Provisional Application No. 63/076,690, filed Sep. 10, 2020, which is incorporated herein by reference.

### SEQUENCE LISTING

The instant application contains a Sequence Listing which has been submitted electronically in XML format and is hereby incorporated by reference in its entirety. Said XML copy, created on Jun. 29, 2023, is named 59446-701_301_SL_ST26 format.xml and is 9,305 bytes in size.

### BRIEF SUMMARY

Provided herein are methods for treating infertility in an individual in need thereof comprising: (a) determining by an assay a level of a first bacteria and a level of a miRNA in a sample from the individual; and (b) if the sample has the level of the first bacteria and the level of the miRNA, then administering a therapy to modulate a microbiome of the individual, thereby treating the infertility in the individual. Further provided herein are methods for assessing infertility in an individual in need thereof comprising: (a) determining by an assay a level of a first bacteria and/or a level of a miRNA in a sample from the individual; and (b) if the sample has the level of the first bacteria and/or the level of the miRNA, then administering a supplementation to modulate a microbiome of the individual, thereby treating the microbiome imbalance in the individual. Further provided herein are methods, wherein the first bacteria is selected from the group consisting of a Proteobacteria, an Actinobacteria, a Bacteroidetes, a Firmicutes, and any combination thereof. Further provided herein are methods, wherein the first bacteria is a species of a Proteobacteria, an Actinobacteria, a Bacteroidetes, or a Firmicutes. Further provided herein are methods, wherein the first bacteria is selected from the group consisting of Lactobacillus iners, Lactobacillus brevis, a Firmicutes, a Bacteroidetes, and any combination thereof. Further provided herein are methods, wherein the method further comprises determining a level of a second bacteria. Further provided herein are methods, wherein the second bacteria is selected from the group consisting of a Proteobacteria, an Actinobacteria, a Bacteroidetes, a Firmicutes, and any combination thereof. Further provided herein are methods, wherein the second bacteria is a species of a Proteobacteria, an Actinobacteria, a Bacteroidetes, or a Firmicutes. Further provided herein are methods, wherein the second bacteria is selected from the group consisting of Lactobacillus iners, Lactobacillus brevis, a Firmicutes, a Bacteroidetes, and any combination thereof. Further provided herein are methods, further comprising determining a ratio of the first bacteria to the second bacteria. Further provided herein are methods, wherein the first bacteria is Lactobacillus iners and the second bacteria is Lactobacillus brevis. Further provided herein are methods, wherein the first bacteria is a Firmicutes and the second bacteria is a Bacteroidetes. Further provided herein are methods, wherein the miRNA is derived from a transcriptome of the individual. Further provided herein are methods, wherein the miRNA is selected from the group consisting of miR21-5p, miR155-5p, and any combination thereof. Further provided herein are methods, wherein the level of the first bacteria is decreased below a threshold level of the first bacteria derived from a cohort of control samples. Further provided herein are methods, wherein the level of the first bacteria is decreased by at least about 0.25-fold below the threshold level. Further provided herein are methods, wherein the level of the miRNA is elevated above a threshold level of the miRNA derived from a cohort of control samples. Further provided herein are methods, wherein the level of the miRNA is elevated by at least about 2-fold above the threshold level. Further provided herein are methods, wherein a ratio of the first bacteria to the second bacteria is elevated above a threshold level of a ratio of the first bacteria to the second bacteria derived from a cohort of control samples. Further provided herein are methods, wherein the ratio of the first bacteria to the second bacteria is elevated by at least about 2-fold above the threshold level. Further provided herein are methods, wherein a ratio of the first bacteria to the second bacteria is decreased below a threshold level of a ratio of the first bacteria to the second bacteria derived from a cohort of control samples. Further provided herein are methods, wherein the ratio of the first bacteria to the second bacteria is decreased by at least about 2-fold below the threshold level. Further provided herein are methods, wherein the control samples are obtained from individuals that are fertile. Further provided herein are methods, further comprising determining by an assay a level of an inflammatory biomarker, and wherein the therapy is administered based on the level of the first bacteria, the level of the miRNA, and the level of the inflammatory biomarker. Further provided herein are methods, wherein the inflammatory biomarker is associated with a disease or disorder selected from the group consisting of anemia, hypovitaminosis B, hypovitaminosis D, hypothyroidism, a metabolic syndrome, polycystic ovary syndrome, endometriosis, autoimmunity, and any combination thereof. Further provided herein are methods, wherein the inflammatory biomarker is selected from the group consisting of anti-thyroid peroxidase, anti-antithyroglobulin antibody, anti-nuclear antibody, anti-Saccharomyces cerevisiae antibody IgA, anti-Saccharomyces cerevisiae antibody IgG, and any combination thereof. Further provided herein are methods, further comprising determining the individual's medical history prior to step (b). Further provided herein are methods, wherein the individual's medical history comprises determining a glycidic metabolic component, a lipidic metabolic component, intestinal permeability, or body mass index of the individual. Further provided herein are methods, wherein the therapy comprises administering a nutritional plan to the individual. Further provided herein are methods, wherein the therapy comprises administering a vitamin, a supplement, a probiotic, or any combination thereof to the individual. Further provided herein are methods, wherein the vitamin is selected from the group consisting of vitamin A, vitamin B12, vitamin C, vitamin D3, vitamin E, and any combination thereof. Further provided herein are methods, wherein the probiotic is selected from the group consisting of Bifidobacterium longum, Bifidobacterium animalis subsp lactis, Bifidobacterium breve, Lactobacillus rhamnosus, Lactobacillus brevis, Lactobacillus acidophilus, Lactobacillus casei, and any combination thereof. Further provided herein are methods, wherein the supplement is selected from the group consisting of omega 3, trans-resveratrol, selenium, L-tryptophan, 5-hydroxytryptophan, magnesium, L-glutamine, and any combination thereof. Further provided herein are methods, wherein the nutritional plan is administered for at least one week. Further provided herein are methods, wherein the nutritional plan is administered for at least one month. Further provided herein are methods, wherein the sample is selected from the group consisting of a saliva sample, a buccal sample, a blood sample, a urine sample, an anal sample, a vaginal sample, and any combination thereof. Further provided herein are methods, wherein the sample is selected from the group consisting of an anal sample, a vagina sample, a buccal sample, a blood sample, and any combination thereof. Further provided herein are methods, wherein the assay is selected from the group consisting of quantitative real-time PCR, northern blot, RNA-seq, microarray, ELISA, homogenous protein assays, immunoblot, and mass spectrometry. Further provided herein are methods, wherein the individual is female.

Provided herein are methods for assessing a likelihood of infertility in an individual comprising: (a) determining a level of a first bacteria in a sample derived from the individual; (b) determining a level of a miRNA in the sample; and (c) assessing the likelihood of infertility in the individual based on the level of the first bacteria and the level of the miRNA, wherein the miRNA provides an area under the curve (AUC) value of greater than about 0.8 in a receiver operating characteristic (ROC) curve analysis. Further provided herein are methods, wherein the first bacteria is selected from the group consisting of a Proteobacteria, an Actinobacteria, a Firmicutes, and any combination thereof. Further provided herein are methods, wherein the first bacteria is a species of a Proteobacteria, an Actinobacteria, a Bacteroidetes, or a Firmicutes. Further provided herein are methods, wherein the first bacteria is selected from the group consisting of Lactobacillus iners, Lactobacillus brevis, a Firmicutes, a Bacteroidetes, and any combination thereof. Further provided herein are methods, wherein the method further comprises determining a level of a second bacteria. Further provided herein are methods, wherein the second bacteria is selected from the group consisting of a Proteobacteria, an Actinobacteria, a Firmicutes, and any combination thereof. Further provided herein are methods, wherein the second bacteria is a species of a Proteobacteria, an Actinobacteria, a Bacteroidetes, or a Firmicutes. Further provided herein are methods, wherein the second bacteria is selected from the group consisting of Lactobacillus iners, Lactobacillus brevis, a Firmicutes, a Bacteroidetes, and any combination thereof. Further provided herein are methods, further comprising determining a ratio of the first bacteria to the second bacteria. Further provided herein are methods, wherein the first bacteria is Lactobacillus iners and the second bacteria is Lactobacillus brevis. Further provided herein are methods, wherein the first bacteria is a Firmicutes and the second bacteria is a Bacteroidetes. Further provided herein are methods, wherein the miRNA is derived from a transcriptome of the individual. Further provided herein are methods, wherein the miRNA is selected from the group consisting of miR21-5p, miR155-5p, and any combination thereof. Further provided herein are methods, wherein the level of the first bacteria is decreased below a threshold level of the first bacteria derived from a cohort of control samples. Further provided herein are methods, wherein the level of the first bacteria is decreased by at least about 0.25-fold below the threshold level. Further provided herein are methods, wherein the level of the miRNA is elevated above a threshold level of the miRNA derived from a cohort of control samples. Further provided herein are methods, wherein the level of the miRNA is elevated by at least about 2-fold above the threshold level. Further provided herein are methods, wherein a ratio of the first bacteria to the second bacteria is elevated above a threshold level of a ratio of the first bacteria to the second bacteria derived from a cohort of control samples. Further provided herein are methods, wherein the ratio of the first bacteria to the second bacteria is elevated by at least about 2-fold above the threshold level. Further provided herein are methods, wherein a ratio of the first bacteria to the second bacteria is decreased below a threshold level of a ratio of the first bacteria to the second bacteria derived from a cohort of control samples. Further provided herein are methods, wherein the ratio of the first bacteria to the second bacteria is decreased by at least about 2-fold below the threshold level. Further provided herein are methods, wherein the control samples are obtained from individuals that are fertile. Further provided herein are methods, further comprising performing an assay of the sample to determine a level of an inflammatory biomarker, and wherein the assessment is based on the level of the first bacteria, the level of the miRNA, and the level of the inflammatory biomarker. Further provided herein are methods, wherein the inflammatory biomarker is associated with a disease or disorder selected from the group consisting of anemia, hypovitaminosis B, hypovitaminosis D, hypothyroidism, a metabolic syndrome, polycystic ovary syndrome, endometriosis, autoimmunity, and any combination thereof. Further provided herein are methods, wherein the inflammatory biomarker is selected from the group consisting of anti-thyroid peroxidase, anti-antithyroglobulin antibody, anti-nuclear antibody, anti-Saccharomyces cerevisiae antibody IgA, anti-Saccharomyces cerevisiae antibody IgG, and any combination thereof. Further provided herein are methods, further comprising determining the individual's medical history prior to step (c). Further provided herein are methods, wherein the individual's medical history comprises determining a glycidic metabolic component, a lipidic metabolic component, intestinal permeability, or body mass index of the individual. Further provided herein are methods, further comprising, if a determination is made based on the level of the first bacteria and the level of the miRNA, then providing a nutritional plan to the individual. Further provided herein are methods, wherein the nutritional plan comprises administering a vitamin, a supplement, a probiotic, or any combination thereof to the individual. Further provided herein are methods, wherein the vitamin is selected from the group consisting of vitamin A, vitamin B12, vitamin C, vitamin D3, vitamin E, and any combination thereof. Further provided herein are methods, wherein the probiotic is selected from the group consisting of Bifidobacterium longum, Bifidobacterium animalis subsp lactis, Bifidobacterium breve, Lactobacillus rhamnosus, Lactobacillus brevis, Lactobacillus acidophilus, Lactobacillus casei, and any combination thereof. Further provided herein are methods, wherein the supplement is selected from the group consisting of omega 3, trans-resveratrol, selenium, L-tryptophan, magnesium, L-glutamine, and any combination thereof. Further provided herein are methods, wherein the nutritional plan is administered for at least one week. Further provided herein are methods, wherein the nutritional plan is administered for at least one month. Further provided herein are methods, wherein the sample is selected from the group consisting of a saliva sample, a buccal sample, a blood sample, a urine sample, an anal sample, a vaginal sample, and any combination thereof. Further provided herein are methods, wherein the sample is selected from the group consisting of an anal sample, a vagina sample, a buccal sample, a blood sample, and any combination thereof. Further provided herein are methods, wherein the assay is selected from the group consisting of quantitative real-time PCR, northern blot, RNA-seq, microarray, ELISA, homogenous protein assays, immunoblot, and mass spectrometry. Further provided herein are methods, wherein the individual is female. Further provided herein are methods, wherein the miRNA provides at least about an 80% sensitivity as determined by ROC curve analysis in assessing the likelihood of infertility in the individual. Further provided herein are methods, wherein the miRNA provides an accuracy of at least about 80% as determined by ROC curve analysis in assessing the likelihood of infertility in the individual.

Provided herein are kits comprising: (a) one or more probes that bind to a first bacteria; (b) one or more probes that bind to a miRNA; (c) a first detecting reagent for detecting binding of the one or more probes to the first bacteria; (d) a second detecting reagent for detecting binding of the one or more probes to the miRNA; and (e) instructions for use. Further provided herein are kits, wherein the first bacteria is selected from the group consisting of a Proteobacteria, an Actinobacteria, a Firmicutes, and any combination thereof. Further provided herein are kits, wherein the first bacteria is a species of a Proteobacteria, an Actinobacteria, a Bacteroidetes, or a Firmicutes. Further provided herein are kits, wherein the first bacteria is selected from the group consisting of Lactobacillus iners, Lactobacillus brevis, a Firmicutes, a Bacteroidetes, and any combination thereof. Further provided herein are kits, wherein the kit further comprises one or more probes for detecting a level of a second bacteria. Further provided herein are kits, wherein the second bacteria is selected from the group consisting of a Proteobacteria, an Actinobacteria, a Firmicutes, and any combination thereof. Further provided herein are kits, wherein the second bacteria is a species of a Proteobacteria, an Actinobacteria, a Bacteroidetes, or a Firmicutes. Further provided herein are kits, wherein the second bacteria is selected from the group consisting of Lactobacillus iners, Lactobacillus brevis, a Firmicutes, a Bacteroidetes, and any combination thereof. Further provided herein are kits, wherein the miRNA is selected from the group consisting of miR21-5p, miR155-5p, and any combination thereof.

Provided herein are methods for treating infertility in an individual in need thereof comprising: (a) determining by an assay a level of a first bacteria, a level of a miRNA, or both in a sample from the individual; and (b) if the sample has the level of the first bacteria, the level of the miRNA, or both, then administering a therapy to modulate a microbiome of the individual, thereby treating the infertility in the individual. Further provided herein are methods for treating infertility in an individual in need thereof wherein step (a) comprises determining by an assay the level of the first bacteria and the level of the miRNA. Further provided herein are methods for treating infertility in an individual in need thereof, wherein the first bacteria is selected from the group consisting of a Proteobacteria, an Actinobacteria, a Bacteroidetes, a Firmicutes, and any combination thereof. Further provided herein are methods for treating infertility in an individual in need thereof, wherein the first bacteria is a species of a Proteobacteria, an Actinobacteria, a Bacteroidetes, or a Firmicutes. Further provided herein are methods for treating infertility in an individual in need thereof, wherein the first bacteria is selected from the group consisting of Lactobacillus iners, Lactobacillus brevis, a Firmicutes, a Bacteroidetes, a Proteobacteria, an Actinobacteria, and any combination thereof. Further provided herein are methods for treating infertility in an individual in need thereof, wherein the method further comprises determining a level of a second bacteria. Further provided herein are methods for treating infertility in an individual in need thereof, wherein the second bacteria is selected from the group consisting of a Proteobacteria, an Actinobacteria, a Bacteroidetes, a Firmicutes, and any combination thereof. Further provided herein are methods for treating infertility in an individual in need thereof, wherein the second bacteria is a species of a Proteobacteria, an Actinobacteria, a Bacteroidetes, or a Firmicutes. Further provided herein are methods for treating infertility in an individual in need thereof, wherein the second bacteria is selected from the group consisting of Lactobacillus iners, Lactobacillus brevis, a Firmicutes, a Bacteroidetes, a Proteobacteria, an Actinobacteria, and any combination thereof. Further provided herein are methods for treating infertility in an individual in need thereof, further comprising determining a ratio of the first bacteria to the second bacteria. Further provided herein are methods for treating infertility in an individual in need thereof, wherein the first bacteria is Lactobacillus iners and the second bacteria is Lactobacillus brevis. Further provided herein are methods for treating infertility in an individual in need thereof, wherein the first bacteria is a Firmicutes and the second bacteria is a Bacteroidetes. Further provided herein are methods for treating infertility in an individual in need thereof, wherein the miRNA is derived from a transcriptome of the individual. Further provided herein are methods for treating infertility in an individual in need thereof, wherein the miRNA is selected from the group consisting of miR21-5p, miR155-5p, and any combination thereof. Further provided herein are methods for treating infertility in an individual in need thereof, wherein the level of the first bacteria is decreased below a threshold level of the first bacteria derived from a cohort of control samples. Further provided herein are methods for treating infertility in an individual in need thereof, wherein the level of the first bacteria is decreased by at least about 0.25-fold below the threshold level. Further provided herein are methods for treating infertility in an individual in need thereof, wherein the level of the miRNA is elevated above a threshold level of the miRNA derived from a cohort of control samples. Further provided herein are methods for treating infertility in an individual in need thereof, wherein the level of the miRNA is elevated by at least about 2-fold above the threshold level. Further provided herein are methods for treating infertility in an individual in need thereof, wherein a ratio of the first bacteria to the second bacteria is elevated above a threshold level of a ratio of the first bacteria to the second bacteria derived from a cohort of control samples. Further provided herein are methods for treating infertility in an individual in need thereof, wherein the ratio of the first bacteria to the second bacteria is elevated by at least about 2-fold above the threshold level. Further provided herein are methods for treating infertility in an individual in need thereof, wherein a ratio of the first bacteria to the second bacteria is decreased below a threshold level of a ratio of the first bacteria to the second bacteria derived from a cohort of control samples. Further provided herein are methods for treating infertility in an individual in need thereof, wherein the ratio of the first bacteria to the second bacteria is decreased by at least about 2-fold below the threshold level. Further provided herein are methods for treating infertility in an individual in need thereof, wherein the control samples are obtained from individuals that are fertile. Further provided herein are methods for treating infertility in an individual in need thereof, further comprising determining by an assay a level of an inflammatory biomarker, and wherein the therapy is administered based on the level of the first bacteria, the level of the miRNA, and the level of the inflammatory biomarker. Further provided herein are methods for treating infertility in an individual in need thereof, wherein the inflammatory biomarker is associated with a disease or disorder selected from the group consisting of anemia, hypovitaminosis B, hypovitaminosis D, hypothyroidism, a metabolic syndrome, polycystic ovary syndrome, endometriosis, autoimmunity, and any combination thereof. Further provided herein are methods for treating infertility in an individual in need thereof, wherein the inflammatory biomarker is selected from the group consisting of anti-thyroid peroxidase, thyroid antithyroglobulin antibody, anti-nuclear antibody, anti-Saccharomyces cerevisiae antibody IgA, anti-Saccharomyces cerevisiae antibody IgG, and any combination thereof. Further provided herein are methods for treating infertility in an individual in need thereof, wherein the therapy is determined in part based on the individual's medical history. Further provided herein are methods for treating infertility in an individual in need thereof, wherein the individual's medical history comprises determining a glycidic metabolic component, a lipidic metabolic component, intestinal permeability, or body mass index of the individual. Further provided herein are methods for treating infertility in an individual in need thereof, wherein the therapy is determined by the level of the first bacteria, a level of the second bacteria, the level of a miRNA, a ratio of the first bacteria to the second bacteria, or a level or presence of a biomarker. Further provided herein are methods for treating infertility in an individual in need thereof, wherein the therapy is selected from a group of predetermined therapies consisting of administering a nutritional plan, administering a vitamin, a supplement, a probiotic, or any combination thereof. Further provided herein are methods for treating infertility in an individual in need thereof, wherein the vitamin is selected from the group consisting of vitamin A, vitamin B12, vitamin C, vitamin D3, vitamin E, and any combination thereof. Further provided herein are methods for treating infertility in an individual in need thereof, wherein the probiotic is selected from the group consisting of Bifidobacterium longum, Bifidobacterium animalis subsp lactis, Bifidobacterium breve, Lactobacillus rhamnosus, Lactobacillus brevis, Lactobacillus acidophilus, Lactobacillus casei, and any combination thereof. Further provided herein are methods for treating infertility in an individual in need thereof, wherein the supplement is selected from the group consisting of omega 3, trans-resveratrol, selenium, L-tryptophan, magnesium, vitamin D, vitamin A, vitamin B12, vitamin E, vitamin C, L-glutamine, and any combination thereof. Further provided herein are methods for treating infertility in an individual in need thereof, wherein the nutritional plan is administered for at least one week. Further provided herein are methods for treating infertility in an individual in need thereof, wherein the nutritional plan is administered for at least one month. Further provided herein are methods for treating infertility in an individual in need thereof, wherein the sample is selected from the group consisting of a saliva sample, a buccal sample, a blood sample, a urine sample, an anal sample, a vaginal sample, and any combination thereof. Further provided herein are methods for treating infertility in an individual in need thereof, wherein the sample is selected from the group consisting of an anal sample, a vagina sample, a buccal sample, a blood sample, and any combination thereof. Further provided herein are methods for treating infertility in an individual in need thereof, wherein the assay is selected from the group consisting of quantitative real-time PCR, northern blot, RNA-seq, microarray, ELISA, homogenous protein assays, immunoblot, and mass spectrometry. Further provided herein are methods for treating infertility in an individual in need thereof, wherein the individual is female. Further provided herein are methods for treating infertility in an individual in need thereof, wherein the individual is male.

Provided herein are methods for assessing a likelihood of infertility in an individual comprising: (a) determining a level of a first bacteria, a level of a miRNA, or both in a sample derived from the individual; and (b) assessing the likelihood of infertility in the individual based on the level of the first bacteria, the level of the miRNA, or both, wherein the miRNA provides an area under the curve (AUC) value of greater than about 0.8 in a receiver operating characteristic (ROC) curve analysis. Provided herein are methods for sample preparation for assessing a likelihood of infertility in an individual comprising: (a) providing a sample from the individual, wherein the sample comprises a first bacteria, a miRNA, or both; (b) lysing the sample thereby producing a lysed sample; (c) performing a reverse transcription reaction on said lysed sample to obtain a lysed, reverse transcribed sample; (d) performing an amplification reaction on the lysed, reverse transcribed sample to obtain an amplified biological sample, wherein said amplification reaction on said lysed, reverse transcribed sample is performed with a set of bacteria primers specific for a bacteria nucleic acid sequence, a set of miRNA primers specific for a miRNA nucleic acid sequence, or both, wherein the bacteria primers specific amplifies the bacteria nucleic acid sequence and the miRNA primers amplifies miRNA nucleic acid sequence; and (e) sequencing the amplified sample using RNA sequencing or quantifying the first bacteria, the miRNA, or both in the amplified sample. Provided herein are methods, wherein the first bacteria is selected from the group consisting of a Proteobacteria, an Actinobacteria, a Firmicutes, and any combination thereof. Further provided herein are methods, wherein the first bacteria is a species of a Proteobacteria, an Actinobacteria, a Bacteroidetes, or a Firmicutes. Further provided herein are methods, wherein the first bacteria is selected from the group consisting of Lactobacillus iners, Lactobacillus brevis, a Firmicutes, a Bacteroidetes, and any combination thereof. Further provided herein are methods, wherein the method further comprises determining a level of a second bacteria. Further provided herein are methods, wherein the second bacteria is selected from the group consisting of a Proteobacteria, an Actinobacteria, a Firmicutes, and any combination thereof. Further provided herein are methods, wherein the second bacteria is a species of a Proteobacteria, an Actinobacteria, a Bacteroidetes, or a Firmicutes. Further provided herein are methods, wherein the second bacteria is selected from the group consisting of Lactobacillus iners, Lactobacillus brevis, a Firmicutes, a Bacteroidetes, and any combination thereof. Further provided herein are methods, further comprising determining a ratio of the first bacteria to the second bacteria. Further provided herein are methods, wherein the first bacteria is Lactobacillus iners and the second bacteria is Lactobacillus brevis. Further provided herein are methods, wherein the first bacteria is a Firmicutes and the second bacteria is a Bacteroidetes. Further provided herein are methods, wherein the miRNA is derived from a transcriptome of the individual. Further provided herein are methods, wherein the miRNA is selected from the group consisting of miR21-5p, miR155-5p, and any combination thereof. Further provided herein are methods, wherein the level of the first bacteria is decreased below a threshold level of the first bacteria derived from a cohort of control samples. Further provided herein are methods, wherein the level of the first bacteria is decreased by at least about 0.25-fold below the threshold level. Further provided herein are methods, wherein the level of the miRNA is elevated above a threshold level of the miRNA derived from a cohort of control samples. Further provided herein are methods, wherein the level of the miRNA is elevated by at least about 2-fold above the threshold level. Further provided herein are methods, wherein a ratio of the first bacteria to the second bacteria is elevated above a threshold level of a ratio of the first bacteria to the second bacteria derived from a cohort of control samples. Further provided herein are methods, wherein the ratio of the first bacteria to the second bacteria is elevated by at least about 2-fold above the threshold level. Further provided herein are methods, wherein a ratio of the first bacteria to the second bacteria is decreased below a threshold level of a ratio of the first bacteria to the second bacteria derived from a cohort of control samples. Further provided herein are methods, wherein the ratio of the first bacteria to the second bacteria is decreased by at least about 2-fold below the threshold level. Further provided herein are methods, wherein the control samples are obtained from individuals that are fertile. Further provided herein are methods, further comprising performing an assay of the sample to determine a level of an inflammatory biomarker. Further provided herein are methods, wherein the assessment is based on the level of the first bacteria, the level of the miRNA, the level of the inflammatory biomarker, or any combination thereof. Further provided herein are methods, wherein the assessment is based on the level of the first bacteria, the level of the miRNA, and the level of the inflammatory biomarker. Further provided herein are methods, wherein the inflammatory biomarker is associated with a disease or disorder selected from the group consisting of anemia, hypovitaminosis B, hypovitaminosis D, hypothyroidism, a metabolic syndrome, polycystic ovary syndrome, endometriosis, autoimmune disorder, and any combination thereof. Further provided herein are methods, wherein the autoimmune disorder is selected from the group consisting of Celiac disease, Hashimoto's disease, Crohn's disease, autoimmune diabetes, Lupus, Graves, rheumatoid arthritis, scleroderma, myasthenia gravis, and Sjogren. Further provided herein are methods, wherein the inflammatory biomarker is selected from the group consisting of anti-thyroid peroxidase, thyroid antithyroglobulin antibody, anti-nuclear antibody, anti-Saccharomyces cerevisiae antibody IgA, anti-Saccharomyces cerevisiae antibody IgG, and any combination thereof. Further provided herein are methods, further comprising determining the individual's medical history prior to step (c). Further provided herein are methods, wherein the individual's medical history comprises determining a glycidic metabolic component, a lipidic metabolic component, intestinal permeability, or body mass index of the individual. Further provided herein are methods, further comprising, if a determination is made based on the level of the first bacteria and the level of the miRNA, then providing a nutritional plan to the individual. Further provided herein are methods, wherein the nutritional plan comprises administering a vitamin, a supplement, a probiotic, or any combination thereof to the individual. Further provided herein are methods, wherein the vitamin is selected from the group consisting of vitamin A, vitamin B12, vitamin C, vitamin D3, vitamin E, and any combination thereof. Further provided herein are methods, wherein the probiotic is selected from the group consisting of Bifidobacterium longum, Bifidobacterium animalis subsp lactis, Bifidobacterium breve, Lactobacillus rhamnosus, Lactobacillus brevis, Lactobacillus acidophilus, Lactobacillus casei, and any combination thereof. Further provided herein are methods, wherein the supplement is selected from the group consisting of omega 3, trans-resveratrol, selenium, L-tryptophan, magnesium, vitamin D, vitamin A, vitamin B12, vitamin E, vitamin C, L-glutamine, and any combination thereof. Further provided herein are methods, wherein the nutritional plan is administered for at least one week.

Further provided herein are methods, wherein the nutritional plan is administered for at least one month. Further provided herein are methods, wherein the sample is selected from the group consisting of a saliva sample, a buccal sample, a blood sample, a urine sample, an anal sample, a vaginal sample, and any combination thereof. Further provided herein are methods, wherein the sample is selected from the group consisting of an anal sample, a vagina sample, a buccal sample, a blood sample, and any combination thereof. Further provided herein are methods, wherein the assay is selected from the group consisting of quantitative real-time PCR, northern blot, RNA-seq, microarray, ELISA, homogenous protein assays, immunoblot, and mass spectrometry. Further provided herein are methods, wherein the individual is female. Further provided herein are methods, wherein the miRNA provides at least about an 80% sensitivity as determined by ROC curve analysis in assessing the likelihood of infertility in the individual. Further provided herein are methods, wherein the miRNA provides an accuracy of at least about 80% as determined by ROC curve analysis in assessing the likelihood of infertility in the individual.

Provided herein are kits comprising: (a) one or more probes that bind to a first bacteria, a miRNA, or both; (b) a first detecting reagent for detecting binding of the one or more probes to the first bacteria, the miRNA, or both; and (c) instructions for use. Further provided herein are kits, wherein the first bacteria is selected from the group consisting of a Proteobacteria, an Actinobacteria, a Firmicutes, and any combination thereof. Further provided herein are kits, wherein the first bacteria is a species of a Proteobacteria, an Actinobacteria, a Bacteroidetes, or a Firmicutes. Further provided herein are kits, wherein the first bacteria is selected from the group consisting of Lactobacillus iners, Lactobacillus brevis, a Firmicutes, a Bacteroidetes, and any combination thereof. Further provided herein are kits, wherein the kit further comprises one or more probes for detecting a level of a second bacteria. Further provided herein are kits, wherein the second bacteria is selected from the group consisting of a Proteobacteria, an Actinobacteria, a Firmicutes, and any combination thereof. Further provided herein are kits, wherein the second bacteria is a species of a Proteobacteria, an Actinobacteria, a Bacteroidetes, or a Firmicutes. Further provided herein are kits, wherein the second bacteria is selected from the group consisting of Lactobacillus iners, Lactobacillus brevis, a Firmicutes, a Bacteroidetes, and any combination thereof. Further provided herein are kits, wherein the miRNA is selected from the group consisting of miR21-5p, miR155-5p, and any combination thereof.

Provided herein are kits comprising: (a) a first collection component for collecting a blood sample; (b) a second collection component for collecting a saliva sample; (c) a third collection component for collecting a vaginal sample; and (d) instructions for use. Further provided herein are kits, wherein the blood sample is analyzed for a biomarker selected from the group consisting of LDL, insulin, anti-nuclear antibody (ANA), vitamin D, vitamin B12, secretory IgA, miR155, miR21, and any combination thereof. Further provided herein are kits, wherein the blood sample is analyzed for a biomarker selected from the group consisting of LDL, insulin, anti-nuclear antibody (ANA), vitamin D, vitamin B12, and any combination thereof. Further provided herein are kits, wherein the saliva sample is analyzed for a biomarker selected from the group consisting of LDL, insulin, anti-nuclear antibody (ANA), vitamin D, vitamin B12, secretory IgA, miR155, miR21, and any combination thereof. Further provided herein are kits, wherein the saliva sample is analyzed for secretory IgA. Further provided herein are kits, wherein the vaginal sample is analyzed for a biomarker selected from the group consisting of LDL, insulin, anti-nuclear antibody (ANA), vitamin D, vitamin B12, secretory IgA, miR155, miR21, and any combination thereof. Further provided herein are kits, wherein the vaginal sample is analyzed for a biomarker selected from the group consisting of miR155, miR21, and any combination thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts a schema of the methods described herein.

FIG. 2 depicts bacterial gene count by 16S rRNA sequencing in anal swabs. Data are presented as total gene count [Median (middle line), interquartile range (top and bottom lines)], statistical significance (Unpaired t test) was defined as P<0.05.

FIGS. 3A-3B depict differences in bacterial communities by 16S rRNA sequencing in vaginal and anal swabs. The relative proportion of microorganisms in vaginal (FIG. 3A) and anal (FIG. 3B) swabs. Number of subjects per group: UI women n=48 and fertile women n=20. Data are presented as relative expression values normalized to total reads [Median (middle line), interquartile range (top and bottom lines)], statistical significance (Mann-Whitney U test) was defined as P<0.05.

FIGS. 4A-4D depict expression levels of dysregulated miRNAs identified in the selection cohort for vaginal miR-21 (FIG. 4A), vaginal miR155 (FIG. 4B), anal miR21 (FIG. 4C) and anal miR155 (FIG. 4D). Expression profiles of significantly altered miRNAs identified in vaginal and anal swabs from infertile women. Number of subjects per group: UI women n=48 and fertile women n=20. Data are presented as relative expression values normalized to RNU48/RNU6B [Median (middle line), interquartile range (top and bottom lines)], statistical significance (Mann-Whitney U test or Unpaired t test) was defined as P<0.05. *** designates P≤0.001; **designates P<0.01; *designates P<0.05.

FIGS. 5A-5D depicts diagnostic estimates of miRNAs identified as dysregulated in the selection cohort for vaginal miR-21 (FIG. 5A), for vaginal miR155 (FIG. 5B), for anal miR21 (FIG. 5C), and for anal miR155 (FIG. 5D). ROC curve analysis was performed for each of the miRNAs identified as being dysregulated in the selection cohort and the associated AUC.

FIG. 6 depicts dysbiosis, epithelial disruption and local inflammation.

FIG. 7 illustrates exemplary results from kits described herein.

### DETAILED DESCRIPTION

Various inflammatory conditions have been linked to dysbiosis. These include conditions that affect reproductive health including endometriosis, polycystic ovary syndrome (PCOS), and infertility and related clinical and subclinical conditions. Infertility has been linked to dysbiosis, which increases estrogen levels and stimulates inflammatory activity and growth of ectopic endometriotic foci. Several miRNAs and other biomarkers have been described to be associated with dysbiosis and immune disbalance. Disclosed herein are methods for assessing infertility or assessing the likelihood of infertility, and kits for detecting bacteria and miRNA relating to inflammatory conditions characterized by dysbiosis.

### Certain Terminologies

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

The terms “individual,” “patient,” or “subject” are used interchangeably. None of the terms require or are limited to a situation characterized by the supervision (e.g., constant or intermittent) of a health care worker (e.g., a doctor, a registered nurse, a nurse practitioner, a physician's assistant, an orderly, or a hospice worker). Further, these terms refer to human or animal subjects.

“Treating” or “treatment” refers to both therapeutic treatment and prophylactic or preventative measures, wherein the object is to prevent or slow down (lessen) a targeted pathologic condition or disorder. Those in need of treatment include those already with the disorder, as well as those prone to have the disorder, or those in whom the disorder is to be prevented. For example, a subject or mammal is successfully “treated” for infertility, if, after receiving a therapeutic amount of a therapeutic agent, the subject shows observable and/or measurable reduction or relief of, or absence of one or more symptoms of infertility.

Unless defined otherwise, all technical and scientific terms used herein have the same meaning as commonly understood by one of ordinary skill in the art to which the methods and compositions described herein belong. Although any methods and materials similar or equivalent to those described herein can also be used in the practice or testing of the methods and compositions described herein, representative illustrative methods and materials are now described.

### Methods of the Disclosure

Described herein are methods and kits for determining infertility in an individual. Infertility may involve dysbiosis and overexpression of miRNAs in response to this microbiome imbalance. Disclosed herein are methods for treating infertility or assessing the likelihood of infertility, and kits for detecting bacteria and miRNA relating to infertility.

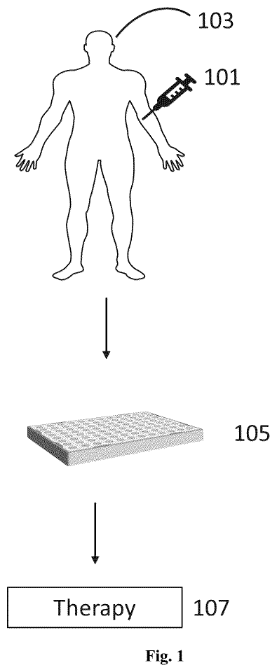

FIG. 1 depicts an exemplary schema of methods described herein. A sample 101 is taken from an individual 103 in need thereof. In some instances, the individual is suspected of being infertile. In some instances, the individual has unexpected infertility. In some instances, the individual has hormone imbalance (e.g., FSH, LH, prolactin), ovarian insufficiency, primary infertility, secondary infertility, oligomenorrhea, or secondary amenorrhea. In some instances, the individual has primary infertility. In some instances, the individual has secondary infertility. In some instances, the individual has had recurrent spontaneous abortion or recurrent pregnancy loss. In some instances, the individual has at least one risk factor for infertility. In some instances, the individual comprises a compromised reproductive potential. In some instances, the sample is selected from a group consisting of a saliva sample, a buccal sample, a blood sample, a urine sample, an anal sample, a vaginal sample, and any combination thereof. In some instances, the sample is selected from the group consisting of an anal sample, a vagina sample, a buccal sample, a blood sample, and any combination thereof. In some instances, a control sample is collected. In some instances, the control is obtained from an individual that is not infertile. In some instances, the individual is a female. The sample 101 is then assayed to determine a level of a first bacteria, a level of a miRNA, or both using an assay 105. In some instances, the first bacteria is Lactobacillus brevis. In some instances, the first bacteria is Lactobacillus iners. In some instances, the first bacteria is selected from the group consisting of Lactobacillus iners, Lactobacillus brevis, a Firmicutes, a Bacteroidetes, a Proteobacteria, an Actinobacteria, and any combination thereof. In some instances, the miRNA is selected from the group consisting of miR21-5p, miR155-5p, and any combination thereof. Depending on the results of the assay 105, a therapy 107 is recommended to the individual. If the sample has the level of the first bacteria and the level of the miRNA, then a therapy 107 is administered to modulate the individual's biome. In some instances, the therapy comprises administering a vitamin, a supplement, a probiotic, and any combination thereof to the individual. In some instances, the vitamin is selected from the group consisting of vitamin A, vitamin B12, vitamin C, vitamin D3, vitamin E, and any combination thereof. In some instances, the probiotic is selected from the group consisting of Bifidobacterium longum, Bifidobacterium animalis subsp lactis, Bifidobacterium breve, Lactobacillus rhamnosus, Lactobacillus brevis, Lactobacillus acidophilus, Lactobacillus casei, and any combination thereof. In some instances, the supplement is selected from the group consisting of omega 3, trans-resveratrol, selenium, L-tryptophan, magnesium, vitamin D, vitamin A, vitamin B12, vitamin E, vitamin C, L-glutamine, and any combination thereof.

Described herein are methods for assessing the likelihood of infertility in an individual. In some instances, the individual has hormone imbalance (e.g., FSH, LH, prolactin), ovarian insufficiency, primary infertility, secondary infertility, oligomenorrhea, or secondary amenorrhea. In some instances, the individual is suspected of infertility. In some instances, the individual has unexpected infertility. In some instances, the individual has primary infertility. In some instances, the individual has secondary infertility. In some instances, the individual has had recurrent spontaneous abortion or recurrent pregnancy loss. In some instances, the individual has at least one risk factor indicating infertility. In some instances, the individual comprises a compromised reproductive potential. In some instances, the individual is female. In some instances, the individual is male. In some instances the individual has anemia, hypovitaminosis B, hypovitaminosis D, a metabolic syndrome, polycystic ovary syndrome (PCOS), endometriosis, hypothyroidism, an autoimmune disorder (e.g., Celiac disease, Hashimoto's disease, Crohn's disease, autoimmune diabetes, Lupus, Graves, rheumatoid arthritis, scleroderma, myasthenia gravis, Sjogren) or other diseases or conditions that directly impacts fertility potential.

Methods as described herein for determining infertility may comprise detecting a bacteria. Various phyla and species of a bacteria can be detected. In some instances, one or more bacteria are detected. In some instances, a plurality of bacteria are detected. Exemplary bacteria belong to any phylum, including an Actinobacteria, a Firmicutes, a Proteobacteria, a Bacteroidetes. In some instances, the species is a Propionibacteria, Staphylococci, Corynebacteria, or Acenitobacteria species.

In some instances, one or more bacteria is detected. In some instances, at least 1, 2, 3, 4, 5, 6, 7, 8, 10, 15, 20, 30, 40, 50, or more than 50 bacteria are detected. In some instances, a first bacteria of the one or more bacteria is a species of Proteobacteria. In some instances, the first bacteria is a species of Actinobacteria. In some instances, the first bacteria is a species of Firmicutes. In some instances, the first bacteria is a species of Allobaculum. In some instances, the first bacteria is a species of Verrucomicrobia. In some instances, the first bacteria is a species of Fusobacteria. In some instances, the first bacteria is a species of Clostridium. In some instances, the first bacteria is a species of Bacteroidetes. In some instances, the first bacteria is a species of bacteria selected from the group consisting of a Proteobacteria, an Actinobacteria, a Bacteroidetes, a Firmicutes, and any combination thereof. In some instances, the first bacteria is Lactobacillus iners. In some instances, the first bacteria is Lactobacillus brevis. In some instances, the first bacteria is selected from the group consisting of Lactobacillus iners, Lactobacillus brevis, a Firmicutes, a Bacteroidetes, a Proteobacteria, an Actinobacteria, and any combination thereof.

Described herein are methods for detecting a level of a miRNA. In some instances, the miRNA is derived from a transcriptome of the individual. In some instances, the miRNA is associated with inflammation. In some instances, the miRNA is associated with tight junctions' disruption. In some instances, the miRNA is associated with exosomes. In some instances, the miRNA is associated with microvesicles. In some instances, the miRNA is expressed in oocytes. In some instances, the miRNA is expressed in embryos. In some instances, the miRNA comprises differential expression depending on the age of an individual.

In some instances, the miRNA is miR21-5p. In some instances, the miRNA is miR155-5p. In some instances, the miRNA is miR-1224. In some instances, the miRNA is miR-2146. In some instances, the miRNA is miR-2134. In some instances, the miRNA is miR-483. In some instances, the miRNA is miR-710. In some instances, the miRNA is miR-2141. In some instances, the miRNA is miR-720. In some instances, the miRNA is miR-34c. In some instances, the miRNA is miR-34c-5p. In some instances, the miRNA is miR-122a. In some instances, the miRNA is miR-146b-5p. In some instances, the miRNA is miR-181a. In some instances, the miRNA is miR-374b. In some instances, the miRNA is miR-509-5p. In some instances, the miRNA is miR-513a-5p. In some instances, the miRNA is miR-193b. In some instances, the miRNA is miR-141. In some instances, the miRNA is miR-9. In some instances, the miRNA is miR-145. In some instances, the miRNA is miR-150. In some instances, the miRNA is miR-212. In some instances, the miRNA is miR-374. In some instances, the miRNA is miR-874. In some instances, the miRNA is miR-20a. In some instances, the miRNA is miR-17-5p. In some instances, the miRNA is miR-106a. In some instances, the miRNA is miR-424. In some instances, the miRNA is miR-199a-5p. In some instances, the miRNA is selected from the group consisting of miR21-5p, miR155-5p, and any combination thereof.

In some instances, the level of the bacteria is elevated above a threshold level of said bacteria derived from a cohort of control samples. In some instances, the elevated level of the bacteria provides an indication of infertility. In some instances, the bacteria is elevated at least about 0.25-fold, at least about 0.5-fold, at least about 1.0-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, or at least about 10-fold above the threshold level of the bacteria derived from a cohort of control samples.

In some instances, the level of the bacteria is decreased below a threshold level of said bacteria derived from a cohort of control samples. In some instances, the decreased level of the bacteria provides an indication of infertility. In some instances, the bacteria is decreased at least about 0.25-fold, at least about 0.5-fold, at least about 1.0-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, or at least about 10-fold below the threshold level of the bacteria derived from a cohort of control samples.

In some instances, the level of the miRNA is elevated above a threshold level of the miRNA derived from a cohort of control samples. In some instances, the level of the miRNA is elevated at least about 0.25-fold, at least about 0.5-fold, at least about 1.0-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, or at least about 10-fold above the threshold level of the miRNA derived from a cohort of control samples.

In some instances, the level of the miRNA is decreased above a threshold level of the miRNA derived from a cohort of control samples. In some instances, the level of the miRNA is decreased at least about 0.25-fold, at least about 0.5-fold, at least about 1.0-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, or at least about 10-fold below the threshold level of the miRNA derived from a cohort of control samples.

The cohort of control samples may be derived from individuals that are fertile. In some instances, the individuals that are fertile comprise individuals with proven fertility. In some instances, the individuals that are fertile comprise individuals who have had at least one healthy baby born in the past 1, 2, 3, 4, 5, or more than 5 years.

In some instances, a level of a second bacteria is determined. In some instances, a second bacteria of the one or more bacteria is a species of Proteobacteria. In some instances, the second bacteria is a species of Actinobacteria. In some instances, the first bacteria is a species of Firmicutes. In some instances, the second bacteria is a species of Allobaculum. In some instances, the second bacteria is a species of Verrucomicrobia. In some instances, the second bacteria is a species of Fusobacteria. In some instances, the second bacteria is a species of Clostridium. In some instances, the bacteria is a species of Bacteroidetes. In some instances, the second bacteria is a species of bacteria selected from the group consisting of a Proteobacteria, an Actinobacteria, a Bacteroidetes, a Firmicutes, and any combination thereof. In some instances, the second bacteria is Lactobacillus iners. In some instances, the second bacteria is Lactobacillus brevis. In some instances, the second bacteria is selected from the group consisting of Lactobacillus iners, Lactobacillus brevis, a Firmicutes, a Bacteroidetes, a Proteobacteria, an Actinobacteria, and any combination thereof.

Methods as described herein may comprise detecting a ratio between a first bacteria and a second bacteria. In some instances, the ratio between the first bacteria and the second bacteria provides an indication of infertility. In some instances, the ratio between the first bacteria and the second bacteria is decreased at least about 0.25-fold, at least about 0.5-fold, at least about 1.0-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, or at least about 10-fold below the threshold level of the ratio between the first bacteria and the second bacteria derived from a cohort of control samples. In some instances, the ratio between the first bacteria and the second bacteria is elevated at least about 0.25-fold, at least about 0.5-fold, at least about 1.0-fold, at least about 1.5-fold, at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, or at least about 10-fold above the threshold level of the ratio between the first bacteria and the second bacteria derived from a cohort of control samples.

In some instances, the methods further comprise determining a level of a biomarker. In some instances, the biomarker is selected from the group consisting of LDL cholesterol, insulin, anti-nuclear antibody (ANA), anti-thyroperoxidase antibody (TPOAb), vitamin D, vitamin B12, secretory IgA, miR155, miR21, and any combination thereof. In some instances, the biomarker is selected from the group consisting of LDL cholesterol, insulin, anti-nuclear antibody (ANA), anti-thyroperoxidase antibody (TPOAb), vitamin D, vitamin B12, secretory IgA, miR155, miR21, and any combination thereof and is measured in the blood. In some instances, the biomarker is selected from the group consisting of LDL, insulin, anti-nuclear antibody (ANA), anti-thyroperoxidase antibody (TPOAb), vitamin D, vitamin B12, and any combination thereof and is measured in the blood. In some instances, the biomarker is selected from the group consisting of LDL, insulin, anti-nuclear antibody (ANA), anti-thyroperoxidase antibody (TPOAb), vitamin D, vitamin B12, secretory IgA, miR155, miR21, and any combination thereof and is measured in a saliva sample. In some instances, the biomarker is secretory IgA and is measured in a saliva sample. In some instances, the biomarker is selected from the group consisting of LDL, insulin, anti-nuclear antibody (ANA), anti-thyroperoxidase antibody (TPOAb), vitamin D, vitamin B12, secretory IgA, miR155, miR21, and any combination thereof and is measured in a vaginal sample. In some instances, the biomarker is selected from the group consisting of LDL, insulin, anti-nuclear antibody (ANA), anti-thyroperoxidase antibody (TPOAb), vitamin D, vitamin B12, secretory IgA, miR155, miR21, and any combination thereof and is measured in a vaginal sample. In some instances, the biomarker is selected from the group consisting of miR155, miR21, and any combination thereof and is measured in a vaginal sample.

In some instances, the methods further comprise determining a level of an inflammatory biomarker. In some instances, the inflammatory biomarker is associated with anemia. In some instances, the inflammatory biomarker is associated with hypovitaminosis B. In some instances, the inflammatory biomarker is associated with hypovitaminosis D. In some instances, the inflammatory biomarker is associated with hypothyroidism. In some instances, the inflammatory biomarker is associated with a metabolic syndrome. In some instances, the inflammatory biomarker is associated with ovulatory and endocrine abnormalities. In some instances, the inflammatory biomarker is associated with polycystic ovary syndrome. In some instances, the inflammatory biomarker is associated with endometriosis. In some instances, the inflammatory biomarker is associated with an autoimmune disease or disorder (e.g., Celiac disease, Hashimoto's disease, Crohn's disease, autoimmune diabetes, Lupus, Graves, rheumatoid arthritis, scleroderma, myasthenia gravis, Sjogren). In some instances, the inflammatory biomarker is associated with the group consisting of anemia, hypovitaminosis B, hypovitaminosis D, hypothyroidism, a metabolic syndrome, polycystic ovary syndrome, endometriosis, an autoimmune disease or disorder, and any combination thereof. In some instances, the inflammatory biomarker is selected from the group consisting of anti-thyroid peroxidase, anti-antithyroglobulin antibody, anti-nuclear antibody, anti-Saccharomyces cerevisiae antibody IgA, anti-Saccharomyces cerevisiae antibody IgG, and any combination thereof. In some instances, an assessment is made based on the level of the first bacteria. In some instances, an assessment is made based on the level of the miRNA. In some instances, an assessment is made based on the level of the inflammatory biomarker. In some instances, an assessment is made based on the level of the first bacteria and the level of the miRNA. In some instances, an assessment is made based on the level of the first bacteria and the level of the inflammatory biomarker. In some instances, an assessment is made based on the level of the miRNA and the level of the inflammatory biomarker. In some instances, an assessment is made based on the level of the first bacteria, the level of the miRNA, and the level of the inflammatory biomarker.

In some instances, the method further comprises determining the individual's medical history. In some instances, the method comprises determining the individual's medical history before therapy. In some instances, the method comprises determining the individual's medical history after therapy. In some instances, the method comprises determining the individual's medical history after detecting a change in the patient's health following therapy. In some instances, the method comprises modifying the therapy based on the patient's medical history. In some instances, the individual's medical history comprises determining a glycidic metabolic component, a lipidic metabolic component, intestinal permeability, or body mass index of the individual.

In some instances, the sample is analyzed for various antibodies. In some instances, the sample is analyzed for thyroid autoantibodies, gastrointestinal autoantibodies, anti-Saccharomyces cerevisiae antibodies, antiphospholipid syndrome antibodies, or anti-nuclear antibodies. In some instances, the sample is analyzed for vitamin levels. In some instances, the sample is analyzed for vitamin D or vitamin B12 levels. In some instances, the sample is analyzed for insulin or glucose levels.

Various assays may be used with the methods described herein. In some cases, the assay is selected from the group consisting of a quantitative real-time PCR, northern blot, RNA-seq, microarray, ELISA, homogenous protein assays, immunoblot, and mass spectrometry. In some embodiments, the assay is an amplification reaction. In some embodiments, the amplification reaction is PCR. In some embodiments, the amplification reaction is quantitative such as quantitative real-time PCR. In some embodiments, the PCR reaction utilizes a TaqMan™ or a similar quantitative PCR technology. In some instances, the assay is quantitative real-time PCR. In some instances, the assay comprises analysis of a nucleic acid molecule, such as sequencing a nucleic acid molecule. Sequencing methods may include whole genome sequencing, next generation sequencing, Sanger-sequencing, 16S rDNA sequencing and 16S rRNA sequencing.

In some instances, the assays detect a nucleic acid. The nucleic acid may comprise DNA, RNA, cDNA, miRNA, mtDNA, single or double-stranded. The nucleic acid can be of any length, as short as oligos of about 5 bp to as long as a megabase or even longer. As used herein, the term “nucleic acid molecule” means DNA, RNA, single-stranded, double-stranded or triple stranded and any chemical modifications thereof. Virtually any modification of the nucleic acid is contemplated. A “nucleic acid molecule” can be of almost any length, from 10, 20, 30, 40, 50, 60, 75, 100, 125, 150, 175, 200, 225, 250, 275, 300, 400, 500, 600, 700, 800, 900, 1000, 1500, 2000, 2500, 3000, 3500, 4000, 4500, 5000, 6000, 7000, 8000, 9000, 10,000, 15,000, 20,000, 30,000, 40,000, 50,000, 75,000, 100,000, 150,000, 200,000, 500,000, 1,000,000, 1,500,000, 2,000,000, 5,000,000 or even more bases in length, up to a full-length chromosomal DNA molecule. For methods that analyze expression of a gene, the nucleic acid isolated from a sample is typically RNA.

In some embodiments, the assay detects a nucleic acid sequence using a primer comprising a sequence TCGTCGGCAGCGTCAGATGTGTATAAGAGACAGGTGCCAGCMGCCGCGGTAA (SEQ ID NO: 1). In some embodiments, the sequence comprises at least or about 70%, 80%, 85%, 90%, 91%, 92%, 93%, 94%, 95%, 96%, 97%, 98%, 99%, or 100% sequence identity to SEQ ID NO: 1. In some instances, the sequence comprises at least or about 95% homology to SEQ ID NO: 1. In some instances, the sequence comprises at least or about 97% homology to SEQ ID NO: 1. In some instances, the sequence comprises at least or about 99% homology to SEQ ID NO: 1. In some instances, the sequence comprises at least or about 100% homology to SEQ ID NO: 1. In some instances, the sequence comprises at least a portion having at least or about 10, 20, 30, 40, or 50, nucleotides of SEQ ID NO: 1.

In some embodiments, the assay detects a nucleic acid sequence using a primer comprising a sequence TCTCGTGGGCTCGGAGATGTGTATAAGAGACAGGGACTACNVGGGTWTCTAAT-3′ (SEQ ID NO: 2). In some embodiments, the sequence comprises at least or about 70%, 80%, 85%, 90%, 91%, 92%, 93%, 94%, 95%, 96%, 97%, 98%, 99%, or 100% sequence identity to SEQ ID NO: 2. In some instances, the sequence comprises at least or about 95% homology to SEQ ID NO: 2. In some instances, the sequence comprises at least or about 97% homology to SEQ ID NO: 2. In some instances, the sequence comprises at least or about 99% homology to SEQ ID NO: 2. In some instances, the sequence comprises at least or about 100% homology to SEQ ID NO: 2. In some instances, the sequence comprises at least a portion having at least or about 10, 20, 30, 40, or 50 nucleotides of SEQ ID NO: 2.

In some instances, the assay comprises using one or more primers or probes that are labeled. In some embodiments, the one or more primers or probes is labeled with an affinity tag. Exemplary affinity tags include, but are not limited to, biotin, desthiobiotin, histidine, polyhistidine, myc, hemagglutinin (HA), FLAG, glutathione S transferase (GST), or derivatives thereof. In some embodiments, the affinity tag is recognized by avidin, streptavidin, nickel, or glutathione.

In some instances, the one or more primers or probes comprises a fluorescent tag. In some embodiments, the fluorescent label is a fluorophore, a fluorescent protein, a fluorescent peptide, quantum dots, a fluorescent dye, a fluorescent material, or variations or combinations thereof.

Exemplary fluorophores include, but are not limited to, Alexa-Fluor dyes (e.g., Alexa Fluor® 350, Alexa Fluor® 405, Alexa Fluor® 430, Alexa Fluor® 488, Alexa Fluor® 500, Alexa Fluor® 514, Alexa Fluor® 532, Alexa Fluor® 546, Alexa Fluor® 555, Alexa Fluor® 568, Alexa Fluor® 594, Alexa Fluor® 610, Alexa Fluor® 633, Alexa Fluor® 647, Alexa Fluor® 660, Alexa Fluor® 680, Alexa Fluor® 700, and Alexa Fluor® 750), APC, Cascade Blue, Cascade Yellow and R-phycoerythrin (PE), DyLight 405, DyLight 488, DyLight 550, DyLight 650, DyLight 680, DyLight 755, DyLight 800, FITC, Pacific Blue, PerCP, Rhodamine, Texas Red, Cy5, Cy5.5, Cy7 and FAM.

Examples of fluorescent peptides include GFP (Green Fluorescent Protein) or derivatives of GFP (e.g., EBFP, EBFP2, Azurite, mKalamal, ECFP, Cerulean, CyPet, YFP, Citrine, Venus, YPet).

Examples of fluorescent dyes include, but are not limited to, xanthenes (e.g., rhodamines, rhodols and fluoresceins, and their derivatives); bimanes; coumarins and their derivatives (e.g., umbelliferone and aminomethyl coumarins); aromatic amines (e.g., dansyl; squarate dyes); benzofurans; fluorescent cyanines; indocarbocyanines; carbazoles; dicyanomethylene pyranes; polymethine; oxabenzanthrane; xanthene; pyrylium; carbostyl; perylene; acridone; quinacridone; rubrene; anthracene; coronene; phenanthrecene; pyrene; butadiene; stilbene; porphyrin; pthalocyanine; lanthanide metal chelate complexes; rare-earth metal chelate complexes; and derivatives of such dyes. In some embodiments, the fluorescein dye is, but not limited to, 5-carboxyfluorescein, fluorescein-5-isothiocyanate, fluorescein-6-isothiocyanate and 6-carboxyfluorescein. In some embodiments, the rhodamine dye is, but not limited to, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, and rhodamine 101 sulfonyl chloride (sold under the tradename of TEXAS RED®). In some embodiments, the cyanine dye is Cy3, Cy3B, Cy3.5, Cy5, Cy5.5, Cy7, IRDYE680, Alexa Fluor 750, IRDye800CW, or ICG.

Fluorescent labels are detected by any suitable method. For example, a fluorescent label is detected by exciting the fluorochrome with the appropriate wavelength of light and detecting the resulting fluorescence, e.g., by microscopy, visual inspection, via photographic film, by the use of electronic detectors such as charge coupled devices (CCDs), or photomultipliers. In some embodiments, the one or more primers or probes are labeled with the same fluorescent label. In some embodiments, the one or more primers or probes are labeled with different fluorescent labels.

Various types of samples may be analyzed using the methods described herein. In some instances, the sample is a biological sample. In some instances, the sample is selected from a group consisting of a saliva sample, a buccal sample, a blood sample, a urine sample, an anal sample, a vaginal sample, an endocervical sample, an endometrial sample, and any combination thereof. In some instances, the sample is selected from the group consisting of an anal sample, a vagina sample, a buccal sample, a blood sample, and any combination thereof.

The sample may be extracted by various methods. In some embodiments, the extraction is done by using swiping, swabbing, tape strips or any other effective microbial collection method. In some embodiments, the sample is from a blood sample and the blood sample is taken, for example, from the individual by a blood draw. In some embodiments, the blood sample is processed by centrifugation such as by density centrifugation. In some embodiments, the blood sample is treated with a red blood cell lysis agent. In some embodiments blood samples are obtained by picking drops of blood from the finger and drying the blood. In some embodiments, the blood sample is analyzed by dried blood spot analysis.

Prior to analysis, the sample may be processed. In some embodiments, DNA is extracted and purified from the biological sample. In some embodiments, RNA is extracted. In some embodiments, RNA is extracted, purified, and reverse transcribed to cDNA. In some embodiments, after RNA or DNA is extracted, the reverse transcribed cDNA or DNA is amplified prior to sequencing. In some embodiments, after RNA or DNA is extracted, the reverse transcribed cDNA or DNA is amplified using quantitative RT-PCR.

Provided herein are biomarkers for assessing the likelihood of infertility in an individual with improved sensitivity, specificity, reliability, and accuracy. In some instances, the biomarkers provide at least about an 80% predictive value as determined by receiver operating characteristic (ROC) curve analysis in assessing the likelihood of infertility in the individual. In some instances, the biomarkers provide at least about an 65%, at least about 70%, at least about 75%, at least about 80%, at least about 85%, at least about 90%, at least about 95%, or more than about 95% predictive value as determined by ROC curve analysis in assessing the likelihood of infertility in the individual. In some instances, the predictive value is a positive predictive value. In some instances, the predictive value is a negative predictive value.

Biomarkers described herein may provide a sensitivity of at least about 80% as determined by ROC curve analysis in assessing the likelihood of infertility in the individual. In some instances, the biomarkers provide a sensitivity of at least about 65%, at least about 70%, at least about 75%, at least about 80%, at least about 85%, at least about 90%, at least about 95%, or more than about 95% as determined by ROC curve analysis in assessing the likelihood of infertility in the individual.

Biomarkers described herein may provide an accuracy of at least about 65%, at least about 70%, at least about 75%, at least about 80%, at least about 85%, at least about 90%, at least about 95%, or more than about 95% as determined by ROC curve analysis in assessing the likelihood of infertility in the individual. In some instances, the accuracy is compared to clinical diagnosis of infertility in an individual. In some instances, the clinical diagnosis of infertility comprises determining a number of failed in vitro fertilization (IVF) treatments. In some instances, an individual is clinically diagnosed as infertile if the individual has had at least 1, 2, 3, 4, 5, 6, or more than 6 failed IVF treatments. In some instances, the individual is clinically diagnosed as infertile if the individual has been diagnosed with unexplained infertility or treated pathology, with more than one year of evolution, either with implantation failures or repeated abortions.

In some instances, the biomarkers provide an area under the curve (AUC) value of greater than about 0.8 in a receiver operating characteristic (ROC) curve analysis. In some instances, the biomarkers provide an area under the curve (AUC) value of at least about 0.6, at least about 0.65, at least about 0.7, at least about 0.75, at least about 0.8, at least about 0.85, at least about 0.9, or at least about 0.95 in a receiver operating characteristic (ROC) curve analysis.

Biomarkers described herein may provide a specificity of at least about 65% as determined by ROC curve analysis in assessing the likelihood of infertility in the individual. In some instances, the biomarkers described herein provide at least about 45%, at least about 50%, at least about 55%, at least about 60%, at least about 65%, at least about 70%, at least about 80%, at least about 90%, or at least about 95% specificity as determined by ROC curve analysis in assessing the likelihood of infertility in the individual.

Disclosed herein are methods for treating infertility in an individual in need thereof comprising: (a) performing an assay or having performed an assay on a sample to determine a level of a first bacteria and a level of a miRNA and (b) if the sample has the level of the first bacteria and the level of the miRNA, then administering a therapy to modulate the microbiome. In some instances, the first bacteria is selected from the group consisting of a Proteobacteria, an Actinobacteria, a Bacteroidetes, a Firmicutes, and any combination thereof. In some instances, the first bacteria is selected from the group consisting of Lactobacillus iners, Lactobacillus brevis, a Firmicutes, a Bacteroidetes, a Proteobacteria, an Actinobacteria, and any combination thereof. In some instances, the miRNA is derived from a transcriptome of the individual. In some instances, the miRNA is selected from the group consisting of miR21-5p, miR155-5p, and any combination thereof. In some instances, the sample is selected from the group consisting of an anal sample, a vagina sample, a buccal sample, a blood sample, a saliva sample and any combination thereof.

In some embodiments, the therapy comprises providing a nutritional plan. In some embodiments, the therapy comprises a nutraceutical combination of biomedical diets, probiotics, and micronutrition. In some instances, the nutritional plan modulates the individual's microbiome. In some instances, the nutritional plan improves antioxidant capacity, repairs the mucosa, or modulates the innate and adaptive immune system.

In some instances, the nutritional plan comprises administering a vitamin, a supplement, a probiotic, and any combination thereof to the individual. In some instances, the vitamin is selected from the group consisting of vitamin A, vitamin B12, vitamin C, vitamin D3, vitamin E, and any combination thereof. In some instances, the probiotic is selected from the group consisting of Bifidobacterium longum, Bifidobacterium animalis subsp lactis, Bifidobacterium breve, Lactobacillus rhamnosus, Lactobacillus brevis, Lactobacillus acidophilus, Lactobacillus casei, and any combination thereof. In some instances, the supplement is selected from the group consisting of omega 3, trans-resveratrol, selenium, L-tryptophan, magnesium, vitamin D, vitamin A, vitamin B12, vitamin E, vitamin C, L-glutamine, and any combination thereof.

In some instances, the biomedical diet is selected from the group consisting of a hypo fermentative diet, a low glycemic index diet, a low saturated fat diet, and any combination thereof.

In some instances, the individual's diet is considered when providing a nutritional plan. In some instances, the individual's metabolic component (glycidic and lipidic); type and degree of intestinal permeability alteration (celiac disease, alterations of the microbiota, gastrointestinal autoimmunity); and the body mass index is considered. In some instances, an individual's co-existing inflammatory or anti-inflammatory factors are considered. In some instances, the environmental factors, pharmacology factors, or previous diagnosis of a disease or disorder is considered. These nutritional profiles can be adapted to be used at the same time, demonstrating the importance of personalization.

In some instances, the nutritional plan is administered for at least one week, at least two weeks, at least three weeks, or at least four weeks. In some instances, the nutritional plan is administered for about one week, about two weeks, about three weeks, or about four weeks. In some instances, the nutritional plan is administered for at least one month, at least two months, at least three months, at least four months, at least five months, at least six months, at least seven months, at least eight months, at least nine months, at least ten months, at least eleven months, or at least twelve months. In some instances, the nutritional plan is administered for about one month, about two months, about three months, about four months, about five months, about six months, about seven months, about eight months, about nine months, about ten months, about eleven months, or about twelve months. In some instances, the nutritional plan is administered for at least one year, at least two years, at least three years, at least four years, or at least five years. In some instances, the nutritional plan is administered for about one year, about two years, about three years, about four years, or about five years.

Described herein are methods of determining the likelihood of infertility. In some instances, the method requires determining a level of a first bacteria in a sample derived from an individual. In some instances, the method requires determining a level of a miRNA in the sample. In some instances, the method requires assessing the likelihood of infertility in the individual based on the level of the first bacteria and the level of the miRNA. In some instances, the method is at least about 50% accurate, at least about 55% accurate, at least about 60% accurate, at least about 65% accurate, at least about 70% accurate, at least about 75% accurate, at least about 80% accurate, at least about 85% accurate, at least about 90% accurate, or at least 95% accurate in assessing the likelihood of infertility in the individual. In some instances, the likelihood or risk of developing infertility is increased by at least or about 15%, 20%, 25%, 30%, 35%, 40%, 45%, 50%, 55%, 60%, 65%, 70%, 75%, 80%, 85%, 90%, 95% or more than 95% when the level of the miRNA or other biomarker is elevated compared to a reference level derived from a cohort of control samples. In some instances, the likelihood or risk of developing infertility is increased by at least or about 1.5×, 2×, 2.5×, 3×, 3.5×, 4.0×, 4.5×, 5×, 6×, 7×, 8×, 9×, 10×, or more than 10× when the level of the miRNA or other biomarker is elevated compared to a reference level derived from a cohort of control samples.

### Kits

Described herein are kits comprising: (a) a first detection reagent for detecting a first bacteria; (b) a second detection reagent for detecting a miRNA; and (c) instructions for use. Further described herein are kits comprising: (a) a detection reagent for detecting a first bacteria or a miRNA; (b) instructions for use. In some instances, the first bacteria is selected from the group consisting of a Proteobacteria, an Actinobacteria, Bacteroidetes, a Firmicutes, and any combination thereof. In some instances, the first bacteria is selected from the group consisting of Lactobacillus iners, Lactobacillus brevis, a Firmicutes, a Bacteroidetes, a Proteobacteria, an Actinobacteria, and any combination thereof. In some instances, the kit further comprises a third detection reagent for detecting a level of a second bacteria. In some instances, the second bacteria is selected from the group consisting of a Proteobacteria, an Actinobacteria, a Bacteroidetes, a Firmicutes, and any combination thereof. In some instances, the second bacteria is selected from the group consisting of Lactobacillus iners, Lactobacillus brevis, a Firmicutes, a Bacteroidetes, a Proteobacteria, an Actinobacteria, and any combination thereof. In some instances, the miRNA is selected from the group consisting of miR21-5p, miR155-5p, and any combination thereof.

Described herein are kits for sample collection. Described herein are kits comprising (a) a first collection component for collecting a blood sample; (b) a second collection component for collecting a saliva sample; (c) a third collection component for collecting a vaginal sample; and (d) instructions for use. Further described herein are kits comprising (a) a first collection component for collecting a sample selected from the group consisting of a blood sample, a saliva sample, a vaginal sample, and any combination thereof; and (b) instructions for use. In some instances, the blood sample is analyzed for a biomarker selected from the group consisting of LDL cholesterol, insulin, anti-nuclear antibody (ANA), TPOAb, vitamin D, vitamin B12, secretory IgA, miR155, miR21, and any combination thereof. In some instances, the blood sample is analyzed for a biomarker selected from the group consisting of LDL, insulin, anti-nuclear antibody (ANA), vitamin D, vitamin B12, and any combination thereof. In some instances, the saliva sample is analyzed for a biomarker selected from the group consisting of LDL, insulin, anti-nuclear antibody (ANA), vitamin D, vitamin B12, secretory IgA, miR155, miR21, and any combination thereof. In some instances, the saliva sample is analyzed for secretory IgA. In some instances, the vaginal sample is analyzed for a biomarker selected from the group consisting of LDL, insulin, anti-nuclear antibody (ANA), vitamin D, vitamin B12, secretory IgA, miR155, miR21, and any combination thereof. In some instances, the vaginal sample is analyzed for a biomarker selected from the group consisting of miR155, miR21, and any combination thereof. In some instances, the sample collections kits comprise various components selected from the group consisting of swabs, collection containers (e.g., conical tubes, eppendorfs), blood sample kit, sample labels, instructions for collection (e.g., saliva sample collection, vaginal sample collection, anal sample collection), instructions for shipping, instructions for use, and any combination thereof. In some instances, the collections containers comprise a buffer.

In some embodiments, kits comprise nucleic acid or polypeptide isolation reagents. In some embodiments, kits comprise one or more primers or probes for hybridization or amplification of a target nucleic acid whose expression profile or activity profile is associated with infertility. In some embodiments, kits include one or more primers or probes for control genes, such as housekeeping genes. In some embodiments, the one or more primers or probes for control genes are used, for example, in ΔCₜ calculations. In some embodiments, the one or more primers or probes is labeled with an enzyme, a radioactive isotope, or a fluorescent label. In some embodiments, the one or more primers or probes is labeled using an affinity tag. Exemplary affinity tags include, but are not limited to, biotin, desthiobiotin, histidine, polyhistidine, myc, hemagglutinin (HA), FLAG, glutathione S transferase (GST), or derivatives thereof. In some embodiments, the affinity tag is recognized by avidin, streptavidin, nickel, or glutathione. In some embodiments, the kit comprises a detecting reagent that binds to the one or more primers or probes. In some embodiments, the detecting reagent comprises a radioactive isotope or a fluorescent label.

In some embodiments, kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. In other embodiments, the containers are formed from a variety of materials such as glass or plastic.

In some embodiments, kits comprise one or more additional containers, each with one or more of various materials (such as reagents, optionally in concentrated form, and/or devices) desirable from a commercial and user standpoint for use as described herein. Non-limiting examples of such materials include, but not limited to, buffers, primers, enzymes, diluents, filters, carrier, package, container, vial and/or tube labels listing contents and/or instructions for use and package inserts with instructions for use. A set of instructions is optionally included. In some embodiments, a label is on or associated with the container. In some embodiments, a label is on a container when letters, numbers or other characters forming the label are attached, molded or etched into the container itself; a label is associated with a container when it is present within a receptacle or carrier that also holds the container, e.g., as a package insert. In some embodiments, a label is used to indicate that the contents are to be used for a specific therapeutic application. In some embodiments, a label also indicates directions for use of the contents, such as in the methods described herein.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Clinical Studies of Vaginal and Anal Swabs to Quantify Microbiota

This example looks at miRNAs and markers related to infertility.

### Materials and Methods

Study population. The study was carried out with a total of 287 infertile women with unexplained infertility (UI) who had no major medical disorders and who were not taking confounding medications (primarily sex steroids, other infertility drugs) participated in the study. Those women fulfilled the following criteria: diagnosis of unexplained infertility or treated pathology, with more than one year of evolution, either with implantation failures or repeated abortions. In these groups, exclusion criteria were considered: the presence of hydrosalpinx, severe endometriosis, antibiotic treatments, and hormonal untreated disorders. The control group included 20 women with proven fertility during the past three years (at least one healthy baby born). Women recruited for the control fertile group met the following criteria: age between 21 and 39 years old, and body mass index equal to or less than 25. Also, exclusion criteria were considered for this group: being pregnant and/or in breastfeeding period, under hormonal treatment, antibiotic, use of devices intrauterine (IUD), personal history of endocrine, autoimmune disease, or abortions. Participation in the study was voluntary and a written informed consent was obtained. The study was approved by the Ethics Review Committee.

Blood samples analysis. Quantification of thyroid and gastrointestinal autoantibodies, anti-Saccharomyces cerevisiae antibodies, antiphospholipid syndrome and anti-nuclear antibodies were determined together with vitamin D and B12, insulin and glucose blood levels following standard protocols in certified clinical laboratories.

Vaginal fluid and anal samples preparation. Two vaginal and anal samples per patient were obtained using a sterile Dacron swab, suspended in 1 mL of RNAlater solution for stabilizing microbial DNA and RNA and stored at −80° C. in individual tubes until processed. For vaginal samples, patients opened the folds of skin at the vaginal opening, inserted the swab 3 to 5 cm into the vagina, moved the swab in several full circles along the vaginal walls for 20 seconds, and immediately inserted the swab into the collection tube. For anal samples, patients inserted the swab 1 to 2 cm into the anal hole, moved the swab in several full circles for 20 seconds, and immediately inserted the swab into the collection tube.

Microbiological studies. In the present investigation conventional agar-based culture methods for vaginal samples, with Giemsa and Gram staining and agar cultures for 72 h in order to evaluate possible infections were used.

Sample processing and DNA extraction. Metagenomic DNA extraction was carried out from 200 μL of the suspension using the Qiamp DNA Mini kit (Qiagen, USA) following manufacturer's instructions, optimizing the final working elution volume for NGS to 50 μL. All DNA samples were stored at −20° C. prior to sequencing.

16S rRNA Library Preparation and Sequencing. Metagenomic DNA samples were quantified using Quant-iT PicoGreen dsDNA assay kit (Invitrogen Corporation, USA) and further processed using the Illumina 16S Sample Preparation Guide with some modifications. Samples were normalized to 5 ng/μL, and then 12.5 ng of DNA were used to amplify the 16S rRNA V4 hypervariable region using a PCR method (20 cycles) with the following primers (Overhang adapter sequence are underlined): 515F: 5′-TCGTCGGCAGCGTCAGATGTGTATAAGAGACAGGTGCCAGCMGCCGCGGTAA-3′ and 806RB: 5′-TCTCGTGGGCTCGGAGATGTGTATAAGAGACAGGGACTACNVGGGTWTCTAAT-3′. Amplicons were purified using the AMPure XP beads (Beckman Coulter Life Sciences, USA), and a second amplification round was performed using 5 μL of DNA and the Nextera XT Index Primers (N7xx and S5XX). After a final purification with AMPure XP beads and quantification, library DNA were pooled, quantified, denatured and loaded into a NextSeq500 platform using the NextSeq System Denature and Dilute Libraries Guide (Illumina Inc., USA). Libraries were sequenced using a 2×150 cycles chemistry.

RNA isolation. Total RNA (including miRNAs) was isolated from each sample using the mirVana miRNA isolation kit (Life Technologies, USA) according to the manufacturer's instructions. The purity (A260/A280) and quantity of extracted RNA were measured using a Nanodrop One spectrophotometer (Thermo Scientific, USA).

cDNA synthesis. cDNA was synthesized using specific predesigned TaqMan RT and the TaqMan microRNA Reverse Transcription Kit (Applied Biosystems, USA) according to the manufacturer's instructions. Reverse transcription reactions were performed in a final volume of 15 μL, and each reaction contained 4 ng of total RNA for vaginal samples and 10 ng of total RNA for anal samples. The reactions were incubated at 16° C. for 30 min, 42° C. for 30 min and 85° C. for 5 min, with a final hold at 4° C. The reverse transcription reactions without RNA template were used as the RT negative control (for potential contamination with genomic DNA).

qRT-PCR analysis. The final reaction volume was 20 μL, containing 1.33 μL of the RT reaction product. Real-time PCR cycling was conducted on a Thermal Cycler C1000 Touch CFX96 Real-Time System (Bio-Rad, USA) with the following parameters: 95° C. for 10 min, followed by 40-45 cycles of 95° C. for 15 sec and 60° C. for 1 min to identify miRNAs. The threshold cycle (Ct)-values were automatically calculated using Bio-Rad CFX Maestro software and fold changes in expression were calculated by the 2-ΔCt method using RNU48 (vaginal samples) and RNU6B (anal samples) as an endogenous control for miRNAs expression (47). All sample-assay combinations were detected in duplicates for individual samples and negative controls were included in each plate.

Statistical and Bioinformatics Analyses. The expression levels of the four selected miRNAs were normalized to endogenous RNU48/RNU6B (Table 1). The relative miRNA quantity in tested samples from control women versus infertile women was calculated separately with the use of the comparative ΔCt method. The Ct reflects the cycle number at which the fluorescence curve generated within a reaction crosses the threshold in qRT-PCR. The ΔCt was calculated by subtracting the Ct values of endogenous control from the Ct values of the miRNA of interest: ΔCt ¼ (CtmiR of interest—CtRNU48/RNU6B). A ΔΔCt was then calculated by subtracting the ΔCt of samples from infertile patients from the ΔCt of the control samples: ΔΔCt ¼ (ΔCtcontrol−Ctpatients). This method determined the change at the expression of a nucleic acid sequence in a test sample relative to the same sequence in a control sample. The fold-change cut-off for miRNAs was calculated by the equation 2-ΔΔCt (48).

TABLE 1
miRNA probe details and sequence information

| | | Accession |
|---|---|---|
| Probe | Mature miRNA sequence | number |
| RNU48 | GATGACCCCAGGTAACTCTGA | NR_002745 |
| | GTGTGTCGCTGATGCCATCAC | |
| | CGCAGCGCTCTGACC | |
| RNU6B | CGCAAGGATGACACGCAAATT | NR_002752 |
| | CGTGAAGCGTTCCATATTTTT | |
| miR-21-5p | UAGCUUAUCAGACUGAUGUUG | MI0000077 |
| | A | |
| miR-155-5p | UUAAUGCUAAUCGUGAUAGGG | MI0000681 |
| | GUU | |
| miR-193b-3p | AACUGGCCCUCAAAGUCCCGC | MIMAT0002819 |
| | U | |
| miR-141-3p | UAACACUGUCUGGUAAAGAUG | MIMAT0000432 |
| | G | |

The ΔCt distribution was compared with control reference values by means of Mann-Whitney's test or unpaired t test according variances evaluation (P values of <0.05 were considered to be significant). Means and ranges of ΔCt values (from minimum to maximum) were established for each miRNA. The presence of outliers was evaluated by the Grubbs' test. The t test and the area under the receiver operating characteristic (ROC) curve (AUC) values for each miRNA to assess its suitability as a single biomarker were computed with the use of GraphPad Prism 8.0 software (GraphPad Software, USA). Differences were significant at P values of <0.05, and AUC value close to 1 indicated a high diagnostic value. Power was calculated for each miRNA in order to determine if it is over 80%.

Primary efficacy analysis was performed by comparing the treatment with respect to the primary outcome of live birth of the same patients using the Pearson's chi-square test.

### Results

Clinical characterization of study groups. Twenty fertile women were recruited as a control group. They were 35 years old in average and met inclusion and exclusion criteria. 287 infertile patients, 40 years old in average, were recruited. 70% had primary infertility, 14% secondary infertility and 16% recurrent spontaneous abortion. Clinical characteristics of the UI patients are shown in Table 2

TABLE 2

| Clinical | UI women | | Fertile women | |
|---|---|---|---|---|
| characterization | n | % | n | % |
| Anemia | 43 | 15.0 | 1 | 5.0 |
| Hypovitaminosis B and/or D | 210 | 73.2 | 2 | 10.0 |
| Hypothyroidism | 147 | 51.2 | 2 | 10.0 |
| Metabolic syndrome | 161 | 56.1 | 0 | 0 |
| Polycystic ovary syndrome | 53 | 18.5 | 0 | 0 |
| Endometriosis | 78 | 27.2 | 0 | 0 |
| Autoimmunity | 188 | 65.5 | 0 | 0 |
| TPO+ | 57 | 19.9 | 0 | 0 |
| TgAb+ | 53 | 18.5 | 0 | 0 |
| ANA+ | 59 | 20.6 | 0 | 0 |
| ASCA (IgA, IgG) | 88 | 30.7 | 1 | 5.0 |

Anemia: hemoglobin<12 g/dL; Hypovitaminosis B: Vitamin B12<200 pg/mL; Hypovitaminosis D: Vitamin D<30 ng/mL; Hypothyroidism: TSH>4 UI/mL; Metabolic syndrome: altered oral glucose tolerance test (OGTT), glucemia>100 mg/dL, insulin>24 mU/L and/or HOMA>3; Polycystic Ovary Syndrome: ultrasound diagnosis, and/or inositol-metformin intake; Endometriosis: laparoscopic diagnosis and/or CA125>35 UI/mL: Autoimmunity diagnosis of Celiac disease, Hashimoto's disease, Crohn's disease, autoimmune diabetes, Lupus, Graves, rheumatoid arthritis, scleroderma, myasthenia gravis, and/or Sjogren; TPO: Anti-Thyroid Peroxidase; TgAb: Thyroid Antithyroglobulin Antibody; ANA: Anti-nuclear Antibody; ASCA: Anti-Saccharomyces cerevisiae antibodies; IgA: Inmunoglobulin A; IgG: Inmunoglobulin G.

Most of the patients referred gastrointestinal symptoms like gastritis, diarrhea, and abdominal pain which together with anemia, hypovitaminosis and gastrointestinal autoimmunity are linked to a leaky gut condition.

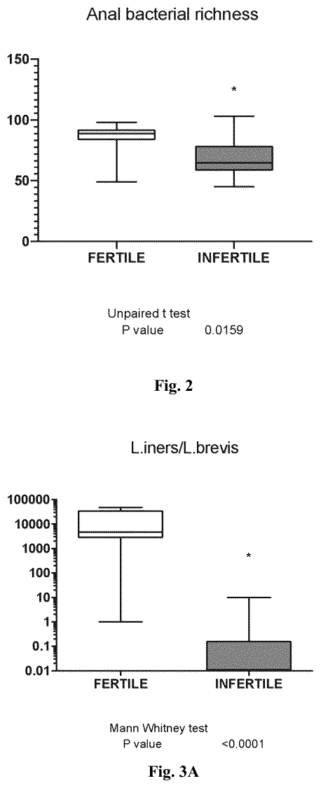

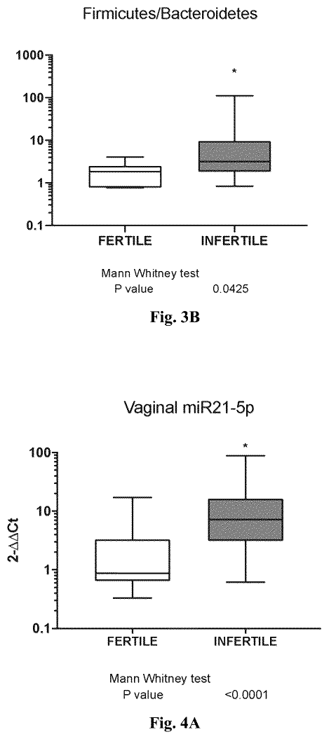

Differences in bacterial communities by 16S rRNA sequencing. A significant differences in bacterial populations was observed. Using a primer set and miSeq platform combination, an average of 49.100 reads were obtained for each sequencing reaction. The general phylogenetic composition at higher taxonomic levels was assessed until a genera and species level was reached where a lower richness at genera level in anal swabs with respect to fertile women was observed. Sixty-nine genera in average were observed in UI patients in comparison to 85 in fertile women (*p<0.05; FIG. 2). Moreover in anal samples, infertile patients showed a difference at the phylum level with a significant increased ratio of Firmicutes/Bacteroidetes (FIG. 3A). There was a significant decrease in the proportion of Lactobacillus iners/Lactobacillus brevis in the infertile group showing a difference at the species level in vaginal samples (FIG. 3B).

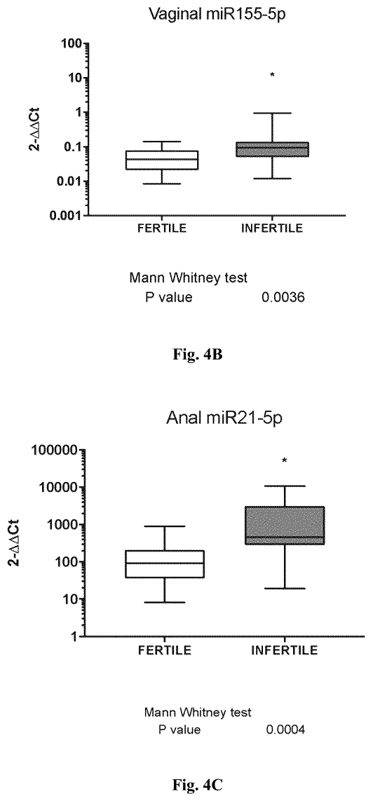

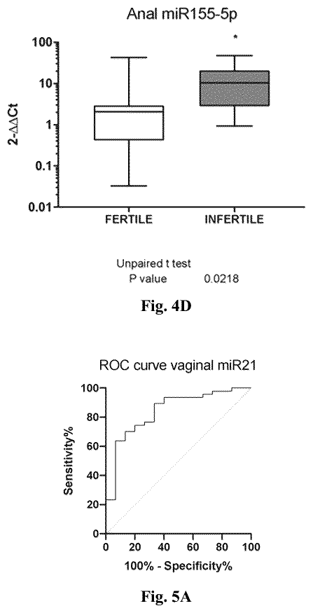

Total miRNAs expressions. In vaginal samples, miR-21-5p (FIG. 4A) was detected, which is associated to tight junctions' disruption, and miR-155-5p (FIG. 4B) which is associated with inflammation was up-regulated in the infertile group (*p<0.05), with AUCs of 0.8426 and 0.8028, respectively. These deregulated miRNAs were also up-regulated in the anal samples (FIGS. 4C-4D, *p<0.05) of the same patients with AUCs of 0.8350 for miR21-5p and 0.8416 for miR155-5p. No statistical difference was observed in miR193b and miR141 in vaginal and/or anal swabs (data not shown).

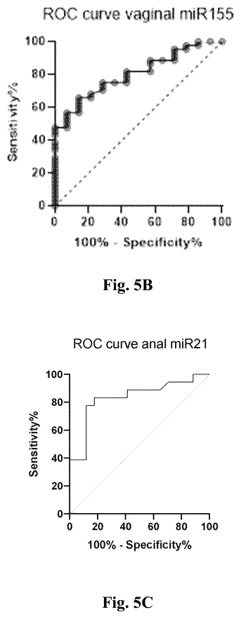

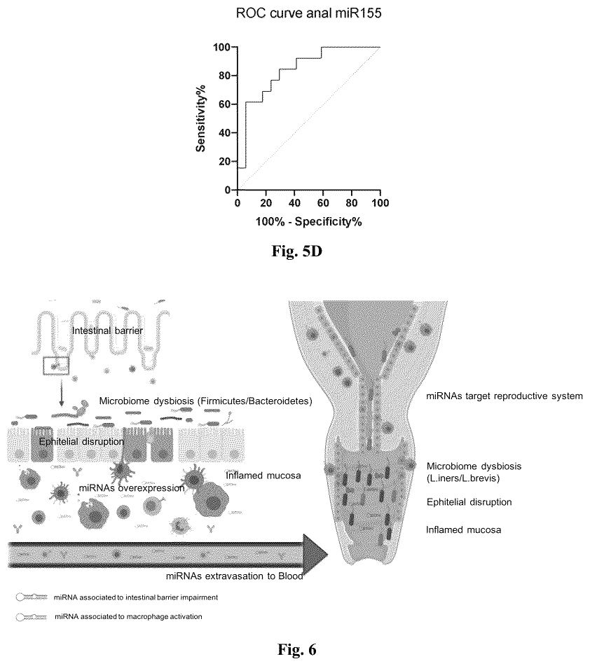

Evaluation of miRNAs as New Biomarkers for Female Infertility. To investigate if those two miRNAs were potential single or combined biomarkers for the assessment of female fertility status, ROC curves were constructed on data from all 68 subjects, including 48 infertile patients compared to 20 control women. ROC curve analysis was used to obtain AUC values that enabled the classification of the predictive power of miRNAs in measurable categories. Only miRNAs that exhibited high AUC values were considered as valid potential biomarkers. ROC curves for miR21-5p and miR155-5p in vaginal and anal samples were constructed (FIGS. 5A-5D) and they showed significant differences between infertile and control group in all cases (Table 3). ROC curves were constructed based on miRNA values and variable fertility. The ROC curves show graphically the connection/trade-off between clinical sensitivity and specificity for every possible cut-off for each miRNA and used to choose the most appropriate cut-off for each test. The cut-off was selected considering the highest true positive rate together with the lowest false positive rate. These miRNAs were found to discriminate against individuals with infertility from normal control subjects with sensitivity over 80%.

TABLE 3

| | Vaginal | Vaginal | | |
|---|---|---|---|---|
| | miR21 | miR155 | Anal miR21 | Anal miR155 |
| AUC | 0.8426 | 0.8028 | 0.8350 | 0.8416 |
| P | <0.0001 | 0.0007 | 0.0007 | 0.0016 |
| Sensitivity (%) | 89.36 | 84.09 | 83.33 | 84.62 |
| Specificity (%) | 66.67 | 75.00 | 82.35 | 70.59 |
| Positive PV | 87.50 | 90.20 | 75.00 | 61.00 |
| Negative PV | 70.6 | 63.16 | 76.00 | 75.00 |
| Power | 80.63 | 80.87 | 82.26 | 80.35 |

Treatment and pregnancy rate. Considering all these parameters and peripheral blood markers of systemic inflammation, patients were classified in 64 different infertile phenotypes and were treated with 53 different combinations of nutraceuticals, probiotics and biomedical diets.

The treatment of the patients was personalized considering coexisting inflammatory and anti-inflammatory factors. Environmental and/or pharmacological factors, and previous diagnosis of underlying pathologies were considered. These factors and especially their evolution during lifetime had an impact on the intestinal, systemic and reproductive immune system. Staggered treatment planning was the most important thing. The clinical causes were treated and once solved, and the effect at the reproductive impact was determined.

In the case of biomedical diets, there are at least 7 nutritional profiles with different requirements, which have been developed taking into account the patient's metabolic component (glycidic and lipidic), type and degree of intestinal permeability alteration (celiac disease, alterations of the microbiota, gastrointestinal autoimmunity) and finally the body mass index. These nutritional profiles were adapted to be used at the same time.

Modulation of the immune system and microbiome was carried out with the combination of different strains of probiotics, and nutraceuticals selected for their antioxidant capacity, repairment of the mucosa and modulators of innate and adaptative immune systems. All of them showed an anti-inflammatory dose specific effect which should be monitored by pre-established controls in order to maintain the delicate immune balance and favor endometrial mucous vascularization and subsequent placentation to achieve a healthy pregnancy.

Of all women in the current analysis, 215 women (75%) got a positive pregnancy test. The clinical conceptions resulted in 129 births (60%) after personalized nutraceutical treatment.

### Conclusion

To evaluate a woman's fertility biome, vaginal and anal swabs to determine microRNA signatures were analyzed. The signature of miRNAs, together with specific blood markers, were observed and the precision nutrients, probiotics and nutraceuticals that were needed to restore fertility was determined. The pregnancy rate in the study population increased from 26% to 65%.

### Example 2. Kits

The kit measures vaginal microRNAs (miRNAs) as a quantitative marker for tight junction disruption and yeast overgrowth (miR21) and macrophage activation and bacterial overgrowth (miR155) and Secretory IgA (SIgA) in saliva samples as a quantitative marker for mucosal immunity. The test measures seven immunometabolism pathways that may help to reduce the risk of dysbiosis by identifying what types of supplements and healthy lifestyle may be the best to choose for a healthier gut condition.

### Kit Components

1 or 2 dacron swabs for vaginal sample collection
1 2 mL greiner tube containing 1mL RNAlater Buffer for vaginal swab transport
Instruction for vaginal swab collection and conservation
1 4 mL sterile greiner tube for saliva sample
Instructions for saliva collection and conservation
1 blood sample kit
Instructions for use
Collected sample labels
Shipping instructions
Shipping labels

### Collection Methods

Samples are collected by the subject and placed in the provided shipping containers and labeled mailers and shipped to the CLIA lab.

Vaginal swab samples: patients should open the folds of skin at the vaginal opening, inserted the swab 3 to 5 cm into the vagina, moved the swab in several full circles along the vaginal walls for 20 seconds, and immediately inserted the swab into the collection tube containing preservation medium (RNAlater) for miRNAs (miR21 and miR155) sample measurements. The collection tube containing the sample is then placed in the plastic specimen bag together with the collected sample label, and then place the bag directly into the box.

Sample should not be collected during menstruation or if the patient has experienced diarrhea in the previous 48 hours.

Patients should not have used antibiotics or vaginal products (except for progesterone ovules) and maintain sexual abstinence for 72 hours prior to collection.

Saliva sample: saliva should be collected directly in the sterile tube without using any swab. Collect the recommended volume of saliva. The recommended volume of saliva to provide is about 2 mL, or about ½ teaspoon. The saliva sample should be just above the fill line of the tube. The transportation supplies for your saliva sample are included in your kit. The collection tube containing the sample is then placed in the plastic specimen bag together with the collected sample label, and then placed the bag directly into the box. Do not eat, drink, smoke, chew gum, brush your teeth, or use mouthwash for at least 30 minutes prior to providing the sample.

Dried Blood Spots: Dried blood spot is a form of collection where patients place blood drops on a filter card after a finger prick with a lancet. Quantification of thyroid autoantibodies, vitamin D, cholesterol LDL and insulin blood levels will be measured by MS/LC in a certified CLIA lab. Once dry, blood spot cards are extremely stable for shipment and storage, and the dried blood format offers excellent correlation with serum tests. Patients can collect their sample at home at the time that suits them. Fasting is recommended (no food or drink other than water) 10-12 hours overnight before collecting in the morning. To encourage blood flow before nicking your finger, rub hands together/swing arm and/or run hand under warm water. Leave the blood spot card open to dry—minimum 4 hours.

Samples collected in the comfort of the patient's home will be delivered to the CLIA laboratory for processing.

### Sample Testing

### MicroRNA Assay in Vaginal Swabs

RNA isolation: Total RNA (including miRNAs) is isolated from each sample using the mirVana miRNA isolation kit (Life Technologies, USA), according to the manufacturer's instructions. The purity (Absorbance 260/280) and quantity of the extracted RNA is measured using a Nanodrop One spectrophotometer (Thermo Scientific, USA).

cDNA synthesis: cDNA is synthesized using specific predesigned TaqMan Reverse Transcription (RT) and the TaqMan microRNA Reverse Transcription Kit (Applied Biosystems, USA), according to the manufacturer's instructions. Reverse transcription reactions are performed in a final volume of 15 μL, and each reaction contains 4 ng of total RNA from the vaginal samples The reactions are incubated at 16° C. for 30 min, 42° C. for 30 min, and 85° C. for 5 min, with a final hold at 4° C. Reverse transcription reactions without an RNA template are used as the RT negative control (for potential contamination with genomic DNA).

qRT-PCR analysis: The final reaction volume is 20 μL, which contains 1.33 μL of the RT reaction product. Real-time PCR cycling is conducted on a Thermal Cycler QuantStudio 6 flex (Applied Biosystems, USA) using the following parameters: 95° C. for 10 min, followed by 40-45 cycles of 95° C. for 15 s, and 60° C. for 1 min to identify the miRNAs. The threshold cycle (Ct) values are automatically calculated using thermal cycler software, and the fold changes in expression are calculated using the 2-ΔΔCt method using RNU48 as endogenous controls for miRNA expression. All sample-assay combinations are detected in duplicates for individual samples, and negative and positive controls are included in each plate.

### Secretory IgA Determination in Saliva Samples

Human IgA will be determined by a sandwich ELISA designed for the quantitative measurement of IgA protein in cell culture supernatant, milk, saliva, serum, urine, and plasma. Quantitate Human IgA with 0.25 ng/ml sensitivity.

ELISA technology employs capture antibodies conjugated to an affinity tag that is recognized by the monoclonal antibody used to coat the plates. This approach to sandwich ELISA allows the formation of the antibody-analyte sandwich complex in a single step, significantly reducing assay time.

Blood Spot Collection Kit: The blood spot samples are processed in accordance with the manufacturer's standard operating procedures.

### Sample Results

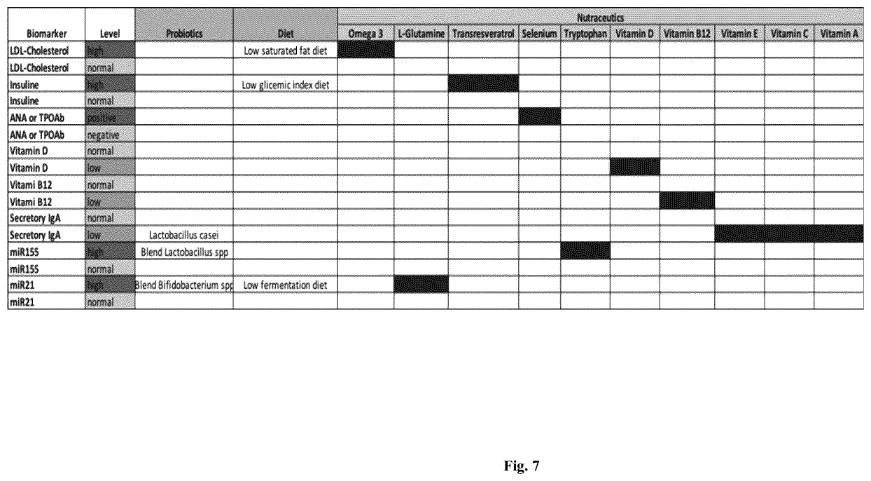

The sample result includes a list of biomarkers and the level detected for each biomarker. As a result of the level detected for each biomarker, the company makes a recommendation for a nutraceutical/probiotic supplement packet and/or other dietary change based on references where it is well understood that such dietary choices may help maintain the level of the biomarker that promotes good health. FIG. 7 illustrates exemplary results.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A method for treating infertility in an individual in need thereof comprising:
(a) determining by an assay a level of a first bacteria, a level of a second bacteria, and/or a level of a miRNA in a sample from the individual;
(b) comparing a ratio of the first bacteria to the second bacteria and/or the level of the miRNA in the sample to a cohort of control samples; and
(c) administering a therapy if the ratio of the first bacteria to the second bacteria and/or the level of the miRNA in the sample is different from the cohort of control samples,
thereby treating the infertility in the individual.

2. (canceled)

3. The method of claim 1, wherein the first bacteria and/or the second bacteria is selected from the group consisting of a Proteobacteria, an Actinobacteria, a Bacteroidetes, a Firmicutes, and any combination thereof.

4. The method of claim 1, wherein the first bacteria and/or the second bacteria is a species of a Proteobacteria, an Actinobacteria, a Bacteroidetes, or a Firmicutes.

5.-10. (canceled)

11. The method of claim 1, wherein the first bacteria is Lactobacillus iners and the second bacteria is Lactobacillus brevis.

12. The method of claim 1, wherein the first bacteria is a Firmicutes and the second bacteria is a Bacteroidetes.

13. The method of claim 1, wherein the miRNA is derived from a transcriptome of the individual, wherein the miRNA is selected from the group consisting of miR21-5p, miR155-5p, and any combination thereof.

14.-16. (canceled)

17. The method of claim 1, wherein the level of the miRNA is elevated above a threshold level of the miRNA derived from the cohort of control samples.

18. (canceled)

19. The method of claim 1, wherein the ratio of the first bacteria to the second bacteria is elevated above a threshold level of a ratio of the first bacteria to the second bacteria derived from the cohort of control samples.

20. (canceled)

21. The method of claim 1, wherein the ratio of the first bacteria to the second bacteria is decreased below a threshold level of a ratio of the first bacteria to the second bacteria derived from the cohort of control samples.

22. (canceled)

23. The method of claim 1, wherein the control samples are obtained from individuals that are fertile

24. The method of claim 1, further comprising determining by an assay a level of an inflammatory biomarker, and wherein the therapy is administered in part based on the level of the inflammatory biomarker.

25. The method of claim 24, wherein the inflammatory biomarker is associated with a disease or disorder selected from the group consisting of anemia, hypovitaminosis B, hypovitaminosis D, hypothyroidism, a metabolic syndrome, polycystic ovary syndrome, endometriosis, autoimmunity, and any combination thereof.

26. The method of claim 24, wherein the inflammatory biomarker is selected from the group consisting of anti-thyroid peroxidase, thyroid antithyroglobulin antibody, anti-nuclear antibody, anti-Saccharomyces cerevisiae antibody IgA, anti-Saccharomyces cerevisiae antibody IgG, and any combination thereof.

27. The method of claim 1, wherein the therapy is determined in part based on the individual's medical history, wherein the medical history comprises a glycidic metabolic component, a lipidic metabolic component, intestinal permeability, or body mass index of the individual.

28. (canceled)

29. (canceled)

30. The method of claim 1, wherein the therapy is selected from a group of predetermined therapies consisting of administering a nutritional plan, administering a vitamin, a supplement, a probiotic, or any combination thereof.

31. The method of claim 30, wherein the vitamin is selected from the group consisting of vitamin A, vitamin B12, vitamin C, vitamin D3, vitamin E, and any combination thereof.

32. The method of claim 30, wherein the probiotic is selected from the group consisting of Bifidobacterium longum, Bifidobacterium animalis subsp lactis, Bifidobacterium breve, Lactobacillus rhamnosus, Lactobacillus brevis, Lactobacillus acidophilus, Lactobacillus casei, and any combination thereof.

33. The method of claim 30, wherein the supplement is selected from the group consisting of omega 3, trans-resveratrol, selenium, L-tryptophan, magnesium, vitamin D, vitamin A, vitamin B12, vitamin E, vitamin C, L-glutamine, and any combination thereof.

34. (canceled)

35. (canceled)

36. The method of claim 1, wherein the sample is selected from the group consisting of a saliva sample, a buccal sample, a blood sample, a urine sample, an anal sample, a vaginal sample, and any combination thereof.

37. (canceled)

38. The method of claim 1, wherein the assay is selected from the group consisting of quantitative real-time PCR, northern blot, RNA-seq, microarray, ELISA, homogenous protein assays, immunoblot, and mass spectrometry.

39.-100. (canceled)


SEQUENCE LISTING (8 sequences)

SEQ ID NO: 1 - DNA, 52 bp, synthetic construct
  misc_feature 1..52: note = Description of Artificial Sequence: Synthetic primer
  source 1..52: mol_type = other DNA
tcgtcggcagcgtcagatgtgtataagagacaggtgccagcmgccgcggtaa

SEQ ID NO: 2 - DNA, 53 bp, synthetic construct
  misc_feature 1..53: note = Description of Artificial Sequence: Synthetic primer
  misc_difference 41
  source 1..53: mol_type = other DNA
tctcgtgggctcggagatgtgtataagagacagggactacnvgggtwtctaat

SEQ ID NO: 3 - DNA, 57 bp, Homo sapiens
  source 1..57: mol_type = genomic DNA
gatgaccccaggtaactctgagtgtgtcgctgatgccatcaccgcagcgctctgacc

SEQ ID NO: 4 - DNA, 42 bp, Homo sapiens
  source 1..42: mol_type = genomic DNA
cgcaaggatgacacgcaaattcgtgaagcgttccatattttt

SEQ ID NO: 5 - RNA, 22 nt, Homo sapiens
  source 1..22: mol_type = other RNA
tagcttatcagactgatgttga

SEQ ID NO: 6 - RNA, 24 nt, Homo sapiens
  source 1..24: mol_type = other RNA
ttaatgctaatcgtgataggggtt

SEQ ID NO: 7 - RNA, 22 nt, Homo sapiens
  source 1..22: mol_type = other RNA
aactggccctcaaagtcccgct

SEQ ID NO: 8 - RNA, 22 nt, Homo sapiens
  source 1..22: mol_type = other RNA
taacactgtctggtaaagatgg